(19) United States
(12) Patent Application Publication
(10) Pub. No.: US 2023/0050395 A1
(43) Pub. Date: Feb. 16, 2023

(54) COMPOSITE BIOMARKERS FOR IMMUNOTHERAPY FOR CANCER

(71) Applicant: Personalis, Inc., Menlo Park, CA (US)

(72) Inventors: Charles Wilbur ABBOTT, III, Menlo Park, CA (US); Sean Michael BOYLE, Menlo Park, CA (US); Rachel Marty PYKE, Menlo Park, CA (US); Eric LEVY, Menlo Park, CA (US); Dattatreya MELLACHERUVU, Menlo Park, CA (US); Rena MCCLORY, Menlo Park, CA (US); Richard CHEN, Menlo Park, CA (US); Robert POWER, Menlo Park, CA (US); Gabor BARTHA, Menlo Park, CA (US); Jason HARRIS, Menlo Park, CA (US); Pamela MILANI, Menlo Park, CA (US); Prateek TANDON, Menlo Park, CA (US); Paul MCNITT, Menlo Park, CA (US); Massimo MORRA, Menlo Park, CA (US); Sejal DESAI, Menlo Park, CA (US); Juan-Sebastian SALVIDAR, Menlo Park, CA (US); Michael CLARK, Menlo Park, CA (US); Christian HAUDENSCHILD, Menlo Park, CA (US); John WEST, Menlo Park, CA (US); Nick PHILLIPS, Menlo Park, CA (US); Simo V. ZHANG, Menlo Park, CA (US)

(73) Assignee: Personalis, Inc., Menlo Park, CA (US)

(21) Appl. No.: 17/965,719

(22) Filed: Oct. 13, 2022

Related U.S. Application Data
(63) Continuation of application No. PCT/US2021/029684, filed on Apr. 28, 2021.
(60) Provisional application No. 63/017,542, filed on Apr. 29, 2020.
(60) Provisional application No. 63/040,943, filed on Jun. 18, 2020.

(51) Int. Cl. G16B 20/00; G16B 5/00; G16B 40/20

(52) U.S. Cl. CPC G16B 20/00; G16B 5/00; G16B 40/20

(57) ABSTRACT

Methods for generating a composite biomarker that identifies a predicted level of responsiveness of a subject to a particular type of an immunotherapy treatment is provided. The method can include generating genomic metrics that represent one or more characteristics corresponding to one or more DNA sequences. The method can also include generating transcriptomic metrics represent one or more characteristics corresponding to a set of peptides that are translated from a corresponding RNA sequence of the one or more RNA sequences. The method can also include generating a composite biomarker score derived from the set of genomic metrics and the set of transcriptomic metrics. The method can also include determining, based on the composite biomarker score, a predicted level of responsiveness of the subject to a particular type of an immunotherapy treatment.

### CROSS-REFERENCES TO RELATED APPLICATIONS

The present application is a continuation of International Application No. PCT/US2021/029684, filed on Apr. 28, 2021, and claims priority to U.S. Provisional Patent Application No. 63/017,542, filed on Apr. 29, 2020, and U.S. Provisional Patent Application No. 63/040,943, filed on Jun. 18, 2020. Each of the applications is hereby incorporated by reference herein in its entirety for all purposes.

### FIELD

This disclosure generally relates to systems and methods for determining composite biomarkers based on genomic and transcriptomic metrics derived from a biological sample. More specifically, but not by way of limitation, this disclosure relates to determining, based on the genomic and transcriptomic metrics, a composite biomarker score that identifies a predicted level of responsiveness of a subject to a particular type of an immunotherapy treatment.

### BACKGROUND

Immunotherapies are used in the treatment of many cancers and autoimmune conditions. While immune checkpoint blockade therapy is known as an effective type of cancer treatment for a variety of malignancies, diagnostic biomarkers that consistently predict subject response to these therapies have remained elusive. Given the highly variable and complex nature of immune-system resistance to immunotherapy, as well as potential toxicities associated with treatment, it can be challenging to accurately predict therapeutic response to certain immunotherapies.

Immunogenomics has emerged as a technique that can determine therapeutic efficacy of immunotherapies. Such technique can lead to a determination of an effective treatment of cancers and may contribute to discovery of several new therapeutics, diagnostics, and processes. For example, immunogenomics can be used to identify neoantigens, which can contribute in the development of precision cancer therapeutics and diagnostics. In addition, genomic data such as variant calls may provide insight into complex immune system responses and resistance to cancer immunotherapies. However, conventional techniques using targeted diagnostic cancer panels provide limited amount of data, which can be unreliable for development of integrative, composite biomarkers.

### BRIEF SUMMARY

In some embodiments, a method and system for determining a composite biomarker score that identifies a predicted level of responsiveness of a subject to a particular type of an immunotherapy treatment is provided. An immunogenomics-analysis system accesses genomic data and transcriptomic data that were generated by processing a biological sample of a subject. In some instances, the biological sample includes one or more cancer cells. The genomic data can identify one or more DNA sequences in the biological sample, in which whole-exome sequencing can be performed to identify the one or more DNA sequences. The transcriptomic data can identify one or more RNA sequences in the biological sample, in which transcriptome sequencing can be used to identify the one or more RNA sequences. Additionally or alternatively, the genomic and the transcriptomic data can be generated from a sample pair that includes the biological sample and a reference biological sample of the subject, in which the reference biological sample does not include the one or more cancer cells.

The immunogenomics-analysis system processes the genomic data to generate a set of genomic metrics. Each of the set of genomic metrics can represent one or more characteristics corresponding to a corresponding DNA sequence the one or more DNA sequences. In some instances, the set of genomic metrics include: (i) a quantitative or categorical metric that represents one or more characteristics for each of one or more somatic mutations in the one or more DNA sequences; (ii) a categorical metric that indicates whether a loss of heterozygosity has occurred in at least one human leukocyte antigen (HLA) gene of the biological sample; and (iii) a quantitative or categorical metric that represents a predicted tumor mutational burden. With respect to the HLA loss of heterozygosity, the corresponding categorical metric can be generated by applying the genomic data to an HLA-deletion-identification machine-learning model.

The immunogenomics-analysis system processes the transcriptomic data to generate a set of transcriptomic metrics. Each of the set of transcriptomic metrics can represent one or more characteristics corresponding to a set of peptides that are translated from a corresponding RNA sequence of the one or more RNA sequences. In some instances, the set of transcriptomic metrics include: (i) a quantitative or categorical metric that represents a predicted neoantigen burden of the biological sample; (ii) a quantitative or categorical metric that represents one or more characteristics of each of one or more candidate neoantigens detected from the biological sample; (iii) a quantitative or categorical metric that represents one or more characteristics of each of one or more HLA proteins for which a loss of cell-surface presentation is detected; (iv) a quantitative or categorical metric that represents one or more characteristics corresponding to an HLA gene that encodes the one or more HLA proteins for which the loss of cell-surface presentation was detected; (v) a quantitative or categorical metric that represents an expression level of a sequence corresponding to an immune cell; and (vi) a quantitative or categorical metric that represents an expression level of one or more T-cell receptors detected from the biological sample. With respect to the HLA proteins for which a loss of cell-surface presentation is detected, the corresponding metric can be generated by applying the genomic and transcriptomic data to a neoantigen-presentation-prediction machine-learning model.

The immunogenomics-analysis system generates a composite biomarker score derived from the set of genomic metrics and the set of transcriptomic metrics and determines, based on the composite biomarker score, a predicted level of responsiveness of the subject to a particular type of an immunotherapy treatment. In some instances, the immunogenomics-analysis system generates the composite biomarker score by: (i) weighting each genomic metric of the set of genomic metrics with a weight value determined based on a corresponding transcriptomic metric of the set of transcriptomic metrics; and (ii) generating the composite biomarker score using the weighted genomic metrics.

The immunogenomics-analysis system outputs a result that corresponds to the predicted level of responsiveness of the subject. The result can be report that identifies, based on the predicted level of responsiveness of the subject to the particular treatment: (i) a treatment recommendation of the particular treatment; (ii) a recommendation to administer the particular treatment to the human subject; and/or (iii) a recommendation to not administer the particular treatment to the human subject. In some embodiments, the recommended treatment is administered to the human subject.

In some embodiments, a computer-program product is provided that is tangibly embodied in a non-transitory machine-readable storage medium and that includes instructions configured to cause one or more data processors to perform part or all of one or more methods disclosed herein.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

Features, embodiments, and advantages of the present disclosure are better understood when the following Detailed Description is read with reference to the following figures. The patent or application file contains at least one drawing executed in color. Copies of this patent or patent application publication with color drawing(s) will be provided by the Office upon request and payment of the necessary fee.

FIG. 1 shows an example of a schematic diagram for generating genomic data and transcriptomic data from a biological sample, according to some embodiments.

FIGS. 2A-B show statistical data corresponding to oncogenic changes in genomic and transcriptomic data corresponding to subjects of a clinical cohort.

FIGS. 3A-C show statistical data corresponding to transcriptomic metrics that identify differentially expressed genes that are associated with immune-system response.

FIG. 4 shows statistical data corresponding to a normalized enrichment score for each differentially regulated immune pathway.

FIGS. 5A-C show statistical data corresponding to transcriptomic metrics that identify expression levels of T-cell receptors.

FIG. 6 shows a set of box plots that identify a comparison of enrichment scores between a first group of responsive subjects and a second group of non-responsive subjects.

FIGS. 7A-B show statistical data corresponding to transcriptomic metrics that identify neoantigen burden across various genes and disease sites.

FIGS. 8A-F show statistical data identifying neoantigen burden scores across various subjects, in which the neoantigen burden score can be predictive of responsiveness of subjects treated with immunotherapies.

FIG. 9A-F show statistical data that identify one or more characteristics relating to mutations present in each subject sample of the discovery cohort.

FIG. 10 shows sets of box plots that identify tumor mutational burden across various driver mutations, disease sites, and subject groups.

FIGS. 11A-D show statistical data identifying composite biomarker scores across various subjects, in which the composite biomarker scores indicate improved performance in predicting responsiveness of subjects treated with immunotherapies.

FIGS. 12A-B show statistical data identifying composite biomarker scores across various subjects, in which the composite biomarker scores indicate improved performance in predicting progression-free and overall survival rates of subjects in the cohort.

FIG. 13A-B show statistical data that identify somatic mutations to HLA genes that may contribute to a decreased probability of neoantigen presentation.

FIG. 14A-B shows examples of sets of panels that identify a comparison of HLA sequences between a normal sample and a corresponding tumor sample of a particular subject.

FIG. 15 includes a flowchart illustrating an example of a method of generating a composite biomarker score, according to some embodiments.

### DETAILED DESCRIPTION

### I. Overview

As described above, efficacy of checkpoint inhibitor therapy can depend on various biological factors, including complex interactions between the tumor, a corresponding tumor microenvironment, and a corresponding immune system. Numerous biomarkers for identifying immune-system responses to immunotherapies have been discussed, including PD-L1 expression, interferon (IFN)-γ based signatures, tumor mutational burden, mismatch repair deficiency, genetic alterations including those within the antigen presenting machinery, HLA loss of heterozygosity, and T-cell repertoire diversity.

As shown by diverse biological factors that can influence the immune-system response to immune checkpoint blockade therapy, there has been increasing effort toward an integrated biomarker that can incorporate various biological factors and accurately predict immune-system response to immunotherapies. For example, conventional techniques have combined information corresponding to immunogenicity and neoantigen clonal structures of a sample to predict the immune-system response to immune checkpoint blockade. The results generated by these conventional techniques have attempted to determine a prognosis in subjects with melanoma, lung cancer, and kidney cancers. While these conventional techniques have yielded somewhat positive results, the conventional techniques still fall short in generating data that can consistently and accurately predict immune-system response. This challenge can be attributed to complex mechanisms that driving immune response to tumors. Moreover, these conventional techniques require large amount of samples from the subject, which can be invasive and difficult to obtain in some circumstances (e.g., age of the subject, subject is pregnant).

To address at least the above deficiencies of conventional systems, the present techniques can be used to determine a composite biomarker score that identifies a predicted level of responsiveness of a subject to a particular type of an immunotherapy treatment. An immunogenomics-analysis system accesses genomic data and transcriptomic data that were generated by processing a biological sample of a subject. In some instances, the biological sample includes one or more cancer cells. The genomic data can identify one or more DNA sequences in the biological sample, in which whole-exome sequencing can be performed to identify the one or more DNA sequences. The transcriptomic data can identify one or more RNA sequences in the biological sample, in which transcriptome sequencing can be used to identify the one or more RNA sequences. Additionally or alternatively, the genomic and the transcriptomic data can be generated from a sample pair that includes the biological sample and a reference biological sample of the subject, in which the reference biological sample does not include the one or more cancer cells.

The immunogenomics-analysis system processes the genomic data to generate a set of genomic metrics. Each of the set of genomic metrics can represent one or more characteristics corresponding to a corresponding DNA sequence the one or more DNA sequences. In some instances, the set of genomic metrics include: (i) a quantitative or categorical metric that represents one or more characteristics for each of one or more somatic mutations in the one or more DNA sequences; (ii) a categorical metric that indicates whether a loss of heterozygosity has occurred in at least one human leukocyte antigen (HLA) gene of the biological sample; and (iii) a quantitative or categorical metric that represents a predicted tumor mutational burden. With respect to the HLA loss of heterozygosity, the corresponding categorical metric can be generated by applying the genomic data to an HLA-deletion-identification machine-learning model.

The immunogenomics-analysis system processes the transcriptomic data to generate a set of transcriptomic metrics. Each of the set of transcriptomic metrics can represent one or more characteristics corresponding to a set of peptides that are translated from a corresponding RNA sequence of the one or more RNA sequences. In some instances, the set of transcriptomic metrics include: (i) a quantitative or categorical metric that represents a predicted neoantigen burden of the biological sample; (ii) a quantitative or categorical metric that represents one or more characteristics of each of one or more candidate neoantigens detected from the biological sample; (iii) a quantitative or categorical metric that represents one or more characteristics of each of one or more HLA proteins for which a loss of cell-surface presentation is detected; (iv) a quantitative or categorical metric that represents one or more characteristics corresponding to an HLA gene that encodes the one or more HLA proteins for which the loss of cell-surface presentation was detected; (v) a quantitative or categorical metric that represents an expression level of a sequence corresponding to an immune cell; and (vi) a quantitative or categorical metric that represents an expression level of one or more T-cell receptors detected from the biological sample. With respect to the HLA proteins for which a loss of cell-surface presentation is detected, the corresponding metric can be generated by applying the genomic and transcriptomic data to a neoantigen-presentation-prediction machine-learning model.

The immunogenomics-analysis system generates a composite biomarker score derived from the set of genomic metrics and the set of transcriptomic metrics and determines, based on the composite biomarker score, a predicted level of responsiveness of the subject to a particular type of an immunotherapy treatment. In some instances, the immunogenomics-analysis system generates the composite biomarker score by: (i) weighting each genomic metric of the set of genomic metrics with a weight value determined based on a corresponding transcriptomic metric of the set of transcriptomic metrics; and (ii) generating the composite biomarker score using the weighted genomic metrics.

The immunogenomics-analysis system outputs a result that corresponds to the predicted level of responsiveness of the subject. The result can be report that identifies, based on the predicted level of responsiveness of the subject to the particular treatment: (i) a treatment recommendation of the particular treatment; (ii) a recommendation to administer the particular treatment to the human subject; and/or (iii) a recommendation to not administer the particular treatment to the human subject. In some embodiments, the recommended treatment is administered to the human subject.

Accordingly, embodiments of the present disclosure provide a technical advantage over conventional techniques by generating a composite biomarker score based on validated, enhanced exome- and transcriptome-based tumor profiling platform. In particular, the composite biomarker score can be determined from metrics that represent characteristics of various tumor and immune-related molecular mechanisms, while minimizing the amount of biological sample used to generate the metrics. Such techniques could improve the accuracy of diagnostic, prognostic and/or treatment recommendations for the corresponding subject, without requiring an invasive procedure of obtaining a large amount of biological samples. Therefore, embodiments of the present disclosure provides a composite immunogenomics framework for accurately predicting a response to immunotherapy treatments by identifying biological mechanisms that drive the response and resistance to such therapies.

While various embodiments of the invention(s) of the present disclosure have been shown and described herein, it will be obvious to those skilled in the art that such embodiments are provided by way of example only. Numerous variations, changes, and substitutions may occur to those skilled in the art without departing from the invention(s). It should be understood that various alternatives to the embodiments of the invention(s) described herein may be employed in practicing any one of the inventions(s) set forth herein.

### II. Definitions

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used herein the term “cancer” or “malignancy” generally refers to a collection of related diseases where the body's cells divide without stopping and spread into surrounding tissues. Cancer can start almost anywhere in the body and develops when the orderly process in removing and replacing old, abnormal, or damaged cells is disrupted, and these cells survive when they should die or new cells form when they are not needed. These cells divide without stopping and are able to spread into and invade both nearby and distant tissues from their origin point.

As used herein, the term “neoantigen” generally refers to newly formed antigens that have not been previously recognized by the immune system. Neoantigens can arise from altered tumor proteins formed as a result of tumor mutations. Neoantigens may constitute the subset of somatic mutations that can be loaded onto MHC class I and class II molecules and presented to T cells. These neoantigens can be seen by the immune system as endogenous tumor-specific (non-self) targets.

As used herein, the term “tumor microenvironment” (tumor microenvironment) refers to the environment around a tumor including the surrounding blood vessels, immune cells, fibroblasts, signaling molecules, and extracellular matrix. A tumor and its microenvironment are closely related and interact constantly with dynamic reciprocity. Tumor progression is influenced by interactions of cancer cells with their environment and shape therapeutic responses and resistance.

As used herein, the term “biomarker” refers to a metabolite or small molecule derived therefrom, that is differentially present (i.e., increased or decreased) in a biological sample from a subject or a group of subjects having a first phenotype (e.g., having a disease) as compared to a biological sample from a subject or group of subjects having a second phenotype (e.g., not having the disease). A biomarker may be differentially present at any level, but is generally present at a level that is increased by at least 5%, by at least I 0%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 100%, by at least 110%, by at least 120%, by at least 130%, by at least 140%, by at least 150%, or more; or is generally present at a level that is decreased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, or by 100% (i.e., absent). A biomarker is preferably differentially present at a level that is statistically significant.

As used herein, the term “level” refers to the level of one or more biomarkers means the absolute or relative amount or concentration of the biomarker in the sample.

As used herein, the term “reference profile” refers to the metabolic profile that is indicative of a healthy subject or one or more of a disease state, condition or body disorder. Within the reference profile, there will be reference levels of one or more biomarkers (metabolites or small molecules derived therefrom) that may be an absolute or relative amount or concentration of the one or more biomarkers, a presence or absence of the one or more biomarkers, a range of amount or concentration of the one or more biomarkers, a minimum and/or maximum amount or concentration of the one or more biomarkers, a mean amount or concentration of the one or more biomarkers, and/or a median amount or concentration of the one or more biomarkers.

As used herein, the term “statistically significant” means at least about a 95% confidence level, preferably at least about a 97% confidence level, more preferably at least about a 98% confidence level and most preferably at least about a 99% confidence level, as determined using parametric or non-parametric statistics, for example, but not limited to ANOVA or Wilcoxon's ranksum Test, wherein the latter is expressed as p<0.05 for at least about a 95% confidence level. [0039] As used herein, the term “immune checkpoint blockade” generally refers to a therapy which focuses on the termination of immune responses by inhibiting immune suppressor molecules thus preventing the termination of immune responses or enabling T-lymphocyte that become exhausted during an immune response.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or I is equivalent to less than or equal to 3, less than or equal to 2, or less than or equal to 1.

The use of the word “a” or “an,” when used in conjunction with the term “comprising” in the claims and/or the specification may mean “one,” but it is also consistent with the meaning of “one or more,” “at least one,” and “one or more than one.”

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

The terms “comprise,” “have,” and “include” are open-ended linking verbs. Any forms or tenses of one or more of these verbs, such as “comprises,” “comprising,” “has,” “having,” “includes,” and “including,” are also open-ended. For example, any method that “comprises,” “has,” or “includes” one or more steps is not limited to possessing only those one or more steps and also covers other unlisted steps.

### III. Immunotherapy Treatments and Immune System Response Mechanisms

A. Tumor Microenvironment

An immune system can detect a wide variety of antigens, such as virus(es), parasitic worm(s), or allergen(s), cancer(s) and initiate a response in the body against foreign substances, abnormal cells and/or tissues. Cancerous growths, including malignant cancerous growths, can also be recognized by the immune cells of a subject and trigger an immune response. The activation of immune cells can trigger numerous intracellular signaling pathways, which require tight control in order to mount an adequate immune response. Cancerous growths can interact intimately with their microenvironment. A tumor may consist not only of a heterogeneous population of cancer cells but also a variety of resident and infiltrating host cells, secreted factors, and extracellular matrix proteins. Cancer and tumor progression may be profoundly influenced by interactions of cancer cells with this tumor microenvironment, which may ultimately determine tumor eradication, metastasis, therapeutic response, or resistance. The mechanisms of the tumor microenvironment on cancer progression may provide a therapeutic avenue in targeting components of the tumor microenvironment, such as in immune checkpoint inhibitor therapies.

The tumor microenvironment, particularly in solid tumors, may remain hostile to immune cells, such as effector T-cells. Barrages of immunosuppressive signals and shortage of essential nutrients within the tumor microenvironment may result in T-cell exhaustion. Overcoming the tumor microenvironment and determining early predictive responses to treatments may an important factor in promoting the efficiency of immunotherapies in eradicating cancer cells in tumors. Metabolic reprogramming and plasticity of cancer cells to adapt to their rapid proliferation may be an important mechanism of treatment resistance in malignant cancers. Several immune cell types are present in the tumor microenvironment and may have an active role in cancer progression, including but not limited to macrophages, B-cells, T-cells, neutrophils, and dendridic cells.

B. Tumor Escape Mechanisms

The progression from neoplastic initiation to malignancy may happen in part because of the failure of immune surveillance. Cancer cells may escape immune recognition and elimination and create an immune-suppressive microenvironment. Due to the high consumption by cancer cells, native immune cells in the region may face a nutrient deprived environment. Multiple metabolic byproducts of cancer cell metabolism such as lactate and the end product of glycolysis may be harmful to the native immune cells, impairing their differentiation, activation, fitness, anti-tumor function, and rendering them broadly unable to compete with the cancer cells.

Metabolic changes in the tumor microenvironment such as hypoxia may also affect the differentiation program of myeloid cells altering their antigen presenting properties. Hypoxia-mediated expression can selectively upregulate the expression of inhibitory ligands promoting T-cell immunosuppression. As cancer-mediated metabolic changes in the tumor microenvironment impact the cellular composition and function of the immune microenvironment, targeting metabolic changes of cancer cells may impact cancer cell growth and progression as well as provide therapeutic targets for improvement of anti-tumor immunity by altering the metabolic program of immune cells and their anti-tumor functions.

C. Immunotherapies

Metabolic processes may regulate immune cell response in quiescent conditions as well as during pathogenic processes such as infection, inflammation, cancer, and autoimmunity. In these complicated conditions, immunotherapies may provide a novel therapeutic avenue. Macrophages as well as other immune cells display metabolic plasticity dependent on disease pathology. Tumor infiltrating lymphocytes may be a notable part of the tumor microenvironment, and correlate with improved prognosis and response to therapy (Cogdill, Andrews, and Wargo 2017 Tomioka et al. 2018).

Immunotherapies may activate the subject's immune system to fight cancer. For effective eradication of cancer cells with immunotherapy, T-cells or other immune cells may recognize tumor peptides presented by human leukocyte antigens (HLAs). The HLA, or major histocompatibility complex may be proteins involved in antigen presentation and can be encoded by HLA genes. Checkpoint inhibitor therapy has demonstrated meaningful antitumor activity, with subject response influenced by a variety of biological factors, including complex interactions between the tumor, tumor microenvironment, and immune system (Hodi et al. 2010; Larkin, Ho and Wolchok 2015 Hugo et al. 2016; Ribas et al. 2016; Wolchok et al. 2017).

Immune checkpoint blockade therapy may be utilized to promote or inhibit T-cell activation. Immune responses may comprise an initiation phase and an activation phase where the immune system recognizes a danger signal and becomes activated by innate signals to fight the danger. This reaction may be one of the first steps for resisting infections and cancer but needs to be turned off once the danger is controlled as persistence of this activation may cause tissue damage. After activation of the immune system a termination phase follows, where endogenous immune suppressor molecules m ay arrest immune responses to prevent damage. In cancer immune therapies, therapeutic approaches classically enhanced the initiation and activation of immune responses to increase the emergence and the efficacy of T-lymphocytes against cancers. Immune checkpoint blockade therapies may focus on the termination of immune responses by inhibiting immune suppressor molecules thus preventing the termination of immune responses or awakening T-lymphocytes that became exhausted during an immune response. Blocking negatively regulating immune checkpoints may restore the capacity of exhausted immune cell s to kill the cancer they infiltrate and drive surviving cancer cells into a state of dormancy.

Immune checkpoints may be co-stimulatory and inhibitory elements intrinsic to the immune system. Immune checkpoints may aid in maintaining self-tolerance and modulating the duration and amplitude of physiological immune responses to prevent injury to tissues when the immune system responds to pathogenic infection. An immune response can also be initiated when a T-cell recognizes antigens that are characteristic of a tumor cell. The equilibrium between the co-stimulatory and inhibitory signals may be used to control the immune response from T-cells can be modulated by immune checkpoint proteins. After T-cells mature and activate in the thymus, T-cells can travel to sites of inflammation and injury to perform repair functions. T-cell function can occur either via direct action or through the recruitment of cytokines and membrane ligands involved in the immune system. The steps involved in T-cell maturation, activation, proliferation, and function can be regulated through co-stimulatory and inhibitory signals, namely through immune checkpoint proteins. Tumors can dysregulate checkpoint protein function as an immune-resistance mechanism. Thus, the development of modulators of checkpoint proteins can have therapeutic value. Non-limiting examples of immune checkpoint molecules include CTLA4 and PD-I. These checkpoint molecules can operate upstream of IL-2 in a pathway.

### IV. Examples of Biomarkers Used for Predicting Immune System Response to Immunotherapies

Immunological checkpoint molecules may be members of the immunoglobulin superfamily and may be inhibitory receptors that prevent uncontrolled immune reactions. The adaptive immune response may be controlled by such checkpoint molecules, which can be used for maintaining self-tolerance and minimizing collateral tissue damage that can occur during an immune response. Numerous biomarkers of response to immune checkpoint blockade have been proposed, including PD-L I expression, interferon (IFN γ based signatures, tumor mutational burden, microsatellite instability (MSI) and mismatch repair deficiency, genetic alterations including those within the antigen presenting machinery (antigen presenting machinery), HLA loss of heterozygosity (HLA loss of heterozygosity), and T cell repertoire diversity (Herbst et al. 2014; Gao et al. 2016; Zaretsky et al. 2016; Roh et al. 2017 Sade-Feldman et al. 2017; Mariathasan et al. 2018; Chowell et al. 2019).

Owing to the diversity of biological features that can influence response to immune checkpoint blockade therapy, there has been increasing effort toward identifying biomarkers that integrate multiple biological features to better predict response to immunotherapy (Charoentong et al. 2017). (Charoentong et al. 2017). In one such effort, a signature combining purity-corrected tumor mutational burden along with receptor tyrosine kinase (RTK) mutations, HLA mutations, and smoking signatures was used to predict immune checkpoint blockade response in non-small-cell lung carcinoma (NSCLC) (Anagnostou et al. 2020), while a melanoma study combined genomic, transcriptomic, and clinical data to predict response to immune checkpoint blockade (Liu et al. 2019).

Neoantigens can constitute the subset of somatic mutations that can be loaded onto MHC class I and class II molecules and presented to T cells. These neoantigens can be seen by the immune system as endogenous tumor-specific (non-self) targets. Immune checkpoint blockade is considered to exploit the ability of cytotoxic (CD8+) T cells to detect and destroy cancer cells displaying neoantigens on their h-IC class I molecules (Schumacher and Schreiber 2015). Work integrating immunogenicity and neoantigen clonal structures predicted response to immune checkpoint blockade and prognosis in subjects with melanoma, lung cancer, and kidney cancers, suggesting broad applicability of the biomarker (Lu et al. 2020).

Recently, an increased effort in identifying surrogate biomarkers for cancer diagnostics and progression using gene expression analyses, metabolomics, and proteomics methods. Gene expression analysis may provide insight on loss of heterozygosity (loss of heterozygosity), a cross-chromosomal event that may result in loss of the entire gene and surrounding chromosomal region, loss of heterozygosity may indicate the absence of a functional tumor suppressor gene in the lost region in cancers. A tumor suppressor gene may be inactivated through either this loss of through a point mutation leaving no tumor suppressor gene to protect the body from cancerous growth. HLA loss of heterozygosity detection may be a pan-cancer biomarker.

### V. Techniques for Generating a Composite Biomarker Score

As described herein, a composite biomarker score generated by an immunogenomics-analysis system can incorporate information pertaining to damaging events in the antigen presentation machinery (e.g., HLA loss of heterozygosity) with predicted neoantigens to stratify subject response to immunotherapy. The composite biomarker score outperforms conventional single-analyte biomarkers, suggesting that complex models capturing multiple aspects of tumor escape can provide more robust stratification of subject response. In addition, such data-intensive biomarkers are clinically practical, with comprehensive tumor profiling in various clinical cohorts achieved using limited tumor tissue. These findings provide an accurate composite biomarker of response in late-stage cancer subjects, as well as evidence supporting the use of whole exome and transcriptome data in a clinical setting.

A. Generating Genomic and Transcriptomic Data

1. Biological Sample

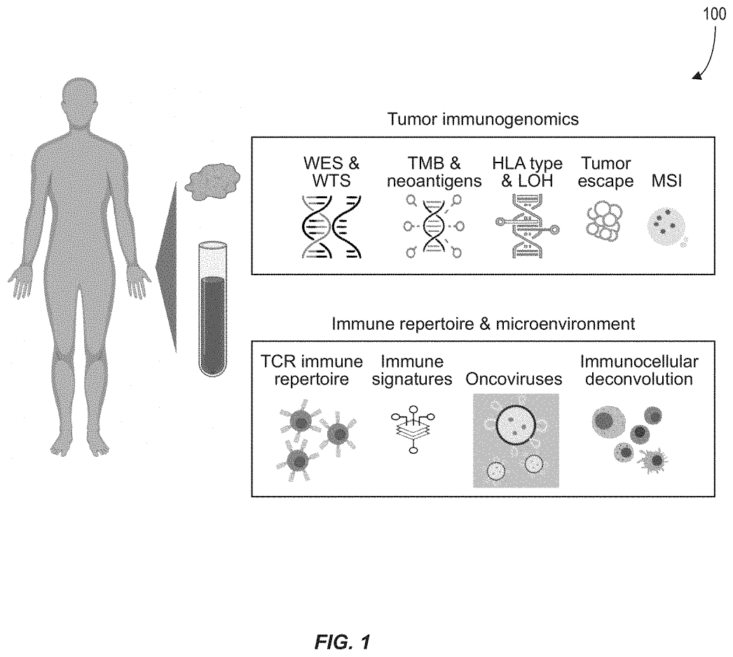

FIG. 1 shows an example of a schematic diagram 100 for generating genomic data and transcriptomic data from a biological sample, according to some embodiments. For example, the schematic diagram 100 includes selecting a biological sample from a subject, in which the biological sample includes cancer cells. In some instances, pre-treatment blood normal and tumor samples are collected from the subject. For example, the pre-treatment blood normal and tumor samples can be collected from a subject with unresectable, stage III/IV melanoma who underwent anti-PD-1 therapy.

The biological sample can be processed to generate an immunogenomics profile of the subject, in which the profile can include comprehensive tumor mutation information, gene expression quantification, neoantigen characterization, HLA (typing, mutation, and loss of heterozygosity), T-cell receptor repertoire profiling, microsatellite instability detection, oncovirus identification, and tumor microenvironment profiling. The profile data can then be analyzed together with clinical outcome, and a composite biomarker score computed for the subject so as to identify the predicted level of responsiveness to a particular immunotherapy treatment.

A sample may be taken from a subject. A sample may be obtained (e.g., extracted or isolated) from or include blood (e.g., whole blood), plasma, serum, umbilical cord blood, chorionic villi, amniotic fluid, lavage fluid (e.g., bronchoalveolar, gastric, peritoneal, ductal, ear, arthroscopic), biopsy sample (e.g., from pre-implantation embryo), celocentesis sample, fetal nucleated cells or fetal cellular remnants, bile, breast milk, urine, saliva, mucosal excretions, sputum, stool, sweat, vaginal fluid, fluid from a hydrocele (e.g., of the testis), vaginal flushing fluids, pleural fluid, ascitic fluid, cerebrospinal fluid, bronchoalveolar lavage fluid, discharge fluid from the nipple, aspiration fluid from different parts of the body (e.g., thyroid, breast), tears, embryonic cells, or fetal cells (e.g., placental cells). In some embodiments, a blood sample is obtained by a heel or finger prick, from scalp veins, or by ear lobe puncture. The biological sample can be a fluid or tissue sample (e.g., skin sample). The biological sample can include any tissue or material derived from a living or dead subject. A biological sample can be a cell-free sample. A biological sample can comprise a protein or nucleic acid (e.g., DNA or RNA r a fragment thereof. A sample may be fixed or may not be fixed. A sample may be embedded or may be free. A sample may be a formalin-fixed paraffin-embedded sample.

The biological sample(s) may include one or more nucleic acid molecules. The nucleic acid molecule may be a DNA molecule, RNA molecule (e.g. mRNA, cRNA or miRNA), and DNA/RNA hybrids. Examples of DNA molecules include, but are not limited to, double-stranded DNA, single-stranded DNA, single-stranded DNA hairpins, cDNA, genomic DNA. The nucleic acid may be an RNA molecule, such as a double-stranded RNA, single-stranded RNA, ncRNA, RNA hairpin, and mRNA. Examples of ncRNA include, but are not limited to, siRNA, miRNA, snoRNA, piRNA, tiRNA, PASR, TASR, aTASR, TSSa-RNA, snRNA, RE-RNA, uaRNA, x-ncRNA, hY RNA, usRNA, snaR, and vtRNA.

2. Sequencing

To generate DNA sequences corresponding to the genomic data from the biological sample, whole exome library preparation and sequencing can be performed. DNA is extracted from the biological sample, processed, and subjected to whole exome sequencing. Whole-exome capture libraries can be constructed using DNA from the tumor and normal blood samples. In some instances, target probes are used to enhance coverage of biomedically and clinically relevant genes. Protocols can be modified to yield an average library insert length of approximately 250 bp. Sequencing reads are subjected to quality control processing (e.g., via FastQC) to provide FASTQ files. FASTQ files are aligned to a reference genome to generate BAM files.

To generate RNA sequences corresponding to the transcriptomic data from the biological sample, transcriptome sequencing can be performed. In some instances, the transcriptome sequencing includes microarrays and RNA-Seq. Microarrays can be configured to measure the abundances of a defined set of transcripts via their hybridization to an array of complementary probes. RNA-Seq can refer to sequencing complementary DNAs of transcripts in the biological samples, in which abundance of the complementary DNAs is derived from the number of counts from each transcript.

In some cases, sample processing includes nucleic acid sample processing and subsequent nucleic acid sample sequencing. Some or all of a nucleic acid sample may be sequenced to provide sequence information, which may be stored or otherwise maintained in an electronic, magnetic or optical storage location. The sequence information may be analyzed with the aid of a computer processor, and the analyzed sequence information may be stored in an electronic storage location. The electronic storage location may include a pool or collection of sequence information and analyzed sequence information generated from the nucleic acid sample.

Some embodiments may include using whole genome sequencing. In some cases, the whole genome sequencing is used to identify variants in a person. In some cases, sequencing can include deep sequencing over a fraction of the genome. For example, the fraction of the genome may be at least about 50; 75; 100; 125; 150; 175; 200; 225; 250; 275; 300; 350; 400; 450; 500; 550; 600; 650; 700; 750; 800; 850; 900; 950; 1,000; 1100; 1200; 1300; 1400; 1500; 1600; 1700; 1800; 1900; 2,000; 3,000; 4,000; 5,000; 6,000; 7,000; 8,000; 9,000; 10,000; 15,000; 20,000; 30,000; 40,000; 50,000; 60,000; 70,000; 80,000; 90,000; 100,000 or more bases or base pairs. In some cases, the genome may be sequenced over 1 million, 2 million, 3 million, 4 million, 5 million, 6 million, 7 million, 8 million, 9 million, 10 million or more than 10 million bases or base pairs. In some cases, the genome may be sequenced over an entire exome (e.g., whole exome sequencing). In some cases, the deep sequencing may include acquiring multiple reads over the fraction of the genome. For example, acquiring multiple reads may include at least 2, 3, 4, 5, 6, 7, 8, 9, 10, 20, 30, 40, 50, 60, 70, 80, 90, 100, 200, 300, 400, 500, 600, 700, 800, 900, 1000, 10,000 reads or more than 10,000 reads over the fraction of the genome.

Some embodiments may include detecting low allelic fractions by deep sequencing. In some cases, the deep sequencing is done by next generation sequencing. In some cases, the deep sequencing is done by avoiding error-prone regions. In some cases, the error-prone regions may include regions of near sequence duplication, regions of unusually high or low % GC, regions of near homopolymers, di- and tri-nucleotide, and regions of near other short repeats. In some cases, the error-prone regions may include regions that lead to DNA sequencing errors (e.g., polymerase slippage in homopolymer sequences).

Some embodiments may include conducting one or more sequencing reactions on one or more nucleic acid molecules in a sample. Some embodiments may include conducting 1 or more, 2 or more, 3 or more, 4 or more, 5 or more, 6 or more, 7 or more, 8 or more, 9 or more, 10 or more, 15 or more, 20 or more, 30 or more, 40 or more, 50 or more, 60 or more, 70 or more, 80 or more, 90 or more, 100 or more, 200 or more, 300 or more, 400 or more, 500 or more, 600 or more, 700 or more, 800 or more, 900 or more, or 1000 or more sequencing reactions on one or more nucleic acid molecules in a sample. The sequencing reactions may be run simultaneously, sequentially, or a combination thereof. The sequencing reactions may include whole genome sequencing or exome sequencing. The sequencing reactions may include Maxim-Gilbert, chain-termination or high-throughput systems. Alternatively, or additionally, the sequencing reactions may include Helioscope™ single molecule sequencing, Nanopore DNA sequencing, Lynx Therapeutics' Massively Parallel Signature Sequencing (MPSS), 454 pyrosequencing, Single Molecule real time (RNAP) sequencing, Illumina (Solexa) sequencing, SOLiD sequencing, Ion Torrent™, Ion semiconductor sequencing, Single Molecule SMRT™ sequencing, Polony sequencing, DNA nanoball sequencing, VisiGen Biotechnologies approach, or a combination thereof. Alternatively, or additionally, the sequencing reactions can include one or more sequencing platforms, including, but not limited to, Genome Analyzer IIx, HiSeq, and MiSeq offered by Illumina, Single Molecule Real Time (SMRT™) technology, such as the PacBio RS system offered by Pacific Biosciences (California) and the Solexa Sequencer, True Single Molecule Sequencing (tSMS™) technology such as the HeliScope™ Sequencer offered by Helicos Inc. (Cambridge, Mass.). Sequencing reactions may also include electron microscopy or a chemical-sensitive field effect transistor (chemFET) array. In some aspects, sequencing reactions include capillary sequencing, next generation sequencing, Sanger sequencing, sequencing by synthesis, sequencing by ligation, sequencing by hybridization, single molecule sequencing, or a combination thereof. Sequencing by synthesis may include reversible terminator sequencing, processive single molecule sequencing, sequential flow sequencing, or a combination thereof. Sequential flow sequencing may include pyrosequencing, pH-mediated sequencing, semiconductor sequencing, or a combination thereof.

Some embodiments may include conducting at least one long read sequencing reaction and at least one short read sequencing reaction. The long read sequencing reaction and/or short read sequencing reaction may be conducted on at least a portion of a subset of nucleic acid molecules. The long read sequencing reaction and/or short read sequencing reaction may be conducted on at least a portion of two or more subsets of nucleic acid molecules. Both a long read sequencing reaction and a short read sequencing reaction may be conducted on at least a portion of one or more subsets of nucleic acid molecules.

Sequencing of the one or more nucleic acid molecules or subsets thereof may include at least about 5; 10; 15; 20; 25; 30; 35; 40; 45; 50; 60; 70; 80; 90; 100; 200; 300; 400; 500; 600; 700; 800; 900; 1,000; 1500; 2,000; 2500; 3,000; 3500; 4,000; 4500; 5,000; 5500; 6,000; 6500; 7,000; 7500; 8,000; 8500; 9,000; 10,000; 25,000; 50,000; 75,000; 100,000; 250,000; 500,000; 750,000; 10,000,000; 25,000,000; 50,000,000; 100,000,000; 250,000,000; 500,000,000; 750,000,000; 1,000,000,000 or more sequencing reads.

Sequencing reactions may include sequencing at least about 50; 60; 70; 80; 90; 100; 110; 120; 130; 140; 150; 160; 170; 180; 190; 200; 210; 220; 230; 240; 250; 260; 270; 280; 290; 300; 325; 350; 375; 400; 425; 450; 475; 500; 600; 700; 800; 900; 1,000; 1500; 2,000; 2500; 3,000; 3500; 4,000; 4500; 5,000; 5500; 6,000; 6500; 7,000; 7500; 8,000; 8500; 9,000; 10,000; 20,000; 30,000; 40,000; 50,000; 60,000; 70,000; 80,000; 90,000; 100,000 or more bases or base pairs of one or more nucleic acid molecules. Sequencing reactions may include sequencing at least about 50; 60; 70; 80; 90; 100; 110; 120; 130; 140; 150; 160; 170; 180; 190; 200; 210; 220; 230; 240; 250; 260; 270; 280; 290; 300; 325; 350; 375; 400; 425; 450; 475; 500; 600; 700; 800; 900; 1,000; 1500; 2,000; 2500; 3,000; 3500; 4,000; 4500; 5,000; 5500; 6,000; 6500; 7,000; 7500; 8,000; 8500; 9,000; 10,000; 20,000; 30,000; 40,000; 50,000; 60,000; 70,000; 80,000; 90,000; 100,000 or more consecutive bases or base pairs of one or more nucleic acid molecules.

Preferably, the sequencing techniques used in the methods of the invention generates at least 100 reads per run, at least 200 reads per run, at least 300 reads per run, at least 400 reads per run, at least 500 reads per run, at least 600 reads per run, at least 700 reads per run, at least 800 reads per run, at least 900 reads per run, at least 1000 reads per run, at least 5,000 reads per run, at least 10,000 reads per run, at least 50,000 reads per run, at least 100,000 reads per run, at least 500,000 reads per run, or at least 1,000,000 reads per run. Alternatively, the sequencing technique used in the methods of the invention generates at least 1,500,000 reads per run, at least 2,000,000 reads per run, at least 2,500,000 reads per run, at least 3,000,000 reads per run, at least 3,500,000 reads per run, at least 4,000,000 reads per run, at least 4,500,000 reads per run, or at least 5,000,000 reads per run.

Preferably, the sequencing techniques used in the methods of the invention can generate at least about 30 base pairs, at least about 40 base pairs, at least about 50 base pairs, at least about 60 base pairs, at least about 70 base pairs, at least about 80 base pairs, at least about 90 base pairs, at least about 100 base pairs, at least about 110, at least about 120 base pairs per read, at least about 150 base pairs, at least about 200 base pairs, at least about 250 base pairs, at least about 300 base pairs, at least about 350 base pairs, at least about 400 base pairs, at least about 450 base pairs, at least about 500 base pairs, at least about 550 base pairs, at least about 600 base pairs, at least about 700 base pairs, at least about 800 base pairs, at least about 900 base pairs, or at least about 1,000 base pairs per read. Alternatively, the sequencing technique used in the methods of the invention can generate long sequencing reads. In some instances, the sequencing technique used in the methods of the invention can generate at least about 1,200 base pairs per read, at least about 1,500 base pairs per read, at least about 1,800 base pairs per read, at least about 2,000 base pairs per read, at least about 2,500 base pairs per read, at least about 3,000 base pairs per read, at least about 3,500 base pairs per read, at least about 4,000 base pairs per read, at least about 4,500 base pairs per read, at least about 5,000 base pairs per read, at least about 6,000 base pairs per read, at least about 7,000 base pairs per read, at least about 8,000 base pairs per read, at least about 9,000 base pairs per read, at least about 10,000 base pairs per read, 20,000 base pairs per read, 30,000 base pairs per read, 40,000 base pairs per read, 50,000 base pairs per read, 60,000 base pairs per read, 70,000 base pairs per read, 80,000 base pairs per read, 90,000 base pairs per read, or 100,000 base pairs per read.

High-throughput sequencing systems may allow detection of a sequenced nucleotide immediately after or upon its incorporation into a growing strand, i.e., detection of sequence in real time or substantially real time. In some cases, high throughput sequencing generates at least 1,000, at least 5,000, at least 10,000, at least 20,000, at least 30,000, at least 40,000, at least 50,000, at least 100,000 or at least 500,000 sequence reads per hour; with each read being at least 50, at least 60, at least 70, at least 80, at least 90, at least 100, at least 120, at least 150, at least 200, at least 250, at least 300, at least 350, at least 400, at least 450, or at least 500 bases per read. Sequencing can be performed using nucleic acids described herein such as genomic DNA, cDNA derived from RNA transcripts or RNA as a template.

3. Alignment

Sequence reads (e.g., the DNA sequences, the RNA sequences) generated by the above sequencing techniques can be mapped to a corresponding reference genome (e.g., hs37d5 reference genome build). In some instances, an alignment pipeline performs alignment, duplicate removal, and base quality score recalibration to generating the genomic and transcriptomic data. The pipeline uses the Picard toolkit (RRID: SCR_006525) for duplicate removal and Genome Analysis Toolkit (GATK, RRID:SCR_001876) to improve sequence alignment and to correct base quality scores (BQSR). Aligned sequence data is then returned in BAM format according to the SAM (RRID:SCR_01095) specification. In some instances, the somatic variants are identified based on the alignment of the sequence reads to the reference genome.

In some instances, whole-transcriptome sequencing was aligned using STAR (RRID:SCR_015899) and normalized expression values in transcripts per million (TPM) was calculated. For RNA sequencing and alignment quality control, the following metrics can be identified: average read length, percentage of uniquely mapped reads, average mapped read pair length, number of splice sites, mismatch rate per base, deletion/insertion rate per base, mean deletion/insertion length, and anomalous read pair alignments including inter-chromosomal and orphaned reads.

B. Transcriptomic Metrics Derived from Transcriptomic Data

The immunogenomics-analysis system processes the transcriptomic data corresponding to the biological sample to generate a set of transcriptomic metrics. Each of the set of transcriptomic metrics can represent one or more characteristics corresponding to a set of peptides that are translated from a corresponding RNA sequence of the one or more RNA sequences. In some instances, the set of transcriptomic metrics include: (i) a quantitative or categorical metric that represents a predicted neoantigen burden of the biological sample; (ii) a quantitative or categorical metric that represents one or more characteristics of each of one or more candidate neoantigens detected from the biological sample; (iii) a quantitative or categorical metric that represents one or more characteristics of each of one or more HLA proteins for which a loss of cell-surface presentation is detected; (iv) a quantitative or categorical metric that represents one or more characteristics corresponding to an HLA gene that encodes the one or more HLA proteins for which the loss of cell-surface presentation was detected; and (v) a quantitative or categorical metric that represents an expression level of one or more T-cell receptors detected from the biological sample. With respect to the HLA proteins for which a loss of cell-surface presentation is detected, the corresponding metric can be generated by applying the genomic and transcriptomic data to a neoantigen-presentation-prediction machine-learning model.

1. Immune Infiltrate Signatures

The set of transcriptomic metrics can include a quantitative or categorical metric that represents an expression level of a sequence corresponding to an immune cell. In some instances, the quantitative or categorical metric is an immune infiltration score, which is derived based on quantities of different types of tumor-infiltrating immune cells. The immune infiltration scores can be calculated using transcriptomic data. For example, semi-quantitative scores representing the enrichment of gene sets can be calculated in single samples. In some instances, a set of reference gene expression signatures representing 17 cell types are used to generate the immune infiltration scores, in which the cell types may include malignant cells, CAFs, endothelial cells, NK cells, B cells, macrophages, and CD8⁺ and CD4⁺ T cells.

To generate the immune infiltration scores, gene set enrichment analysis can be used to compute an enrichment score that is high when the genes specific for a certain cell type are amongst the top highly expressed in the sample of interest (i.e., the cell type is enriched in the sample) and low otherwise. Enrichment scores for the same cell type (gene set) can be compared across samples, profiling immune infiltration for the subject. Additionally or alternatively, the immune infiltration score is generated using deconvolution techniques that can quantitatively estimate the relative fractions of the cell types of interest (e.g., cancer cells). Deconvolution algorithms consider gene expression profiles of a heterogeneous sample as the convolution of the gene expression levels of the different cells, and estimate the unknown cell fractions leveraging on a signature matrix describing the cell-type-specific expression profiles.

2. Expression Levels of T-Cell Receptors

The set of transcriptomic metrics can include a quantitative or categorical metric that represents an expression level of one or more T-cell receptors detected from the biological sample. The expression level of the one or more T-cell receptors can identify a level and distribution of clonal lymphocytes detected in the biological sample. Quality and quantity of lymphocytes from the biological sample can be used to identifying various factors affecting the subject's health and disease. The expression level of the one or more T-cell receptors can be interpreted as having normal immune diversity, development, or reconstitution, or can be otherwise interpreted as having inflammation, infection, vaccination, autoimmunity, or cancer. In some instances, a number of analytic parameters that are used to assess the quality and quantity of a lymphoid infiltrate of the biological sample. The analytic parameters may include diversity, richness, evenness, clonality, and entropy metrics.

In some instances, the expression level of the one or more T-cell receptors corresponds to clonality of T-cell receptor β (TCR-β) sequences detected in the biological sample. The immunogenomics-analysis system processes the transcriptomic data to profile TCR-β clones, which provides augmented (approximately a 100× increase over a standard transcriptome) coverage of TCR-β. Nonproductive clones which have a frame-shift or premature stop codon in the CDR3 sequence can be filtered out, as well as low-confidence clones which have an alignment score below threshold for the V or J hit. Clonality can then calculated as 1-Pielou's evenness.

3. Differential Gene Expressions

The set of transcriptomic metrics can include a quantitative metric that represents read counts per gene identified in the transcriptomic data. For example, counts per million of sequence reads can be calculated by normalizing read counts per gene by the total number of reads identified in the biological sample. In some instances, a threshold is selected as to whether a particular gene should be part of the quantitative metric. For example, only genes with read counts per million >0 in 25% or more of the samples of a cohort can be included for analysis. In some instances, remaining data are processed using rlog transformation and differential gene expression are analyzed. Genes with an adjusted p value <0.05, and a minimum log 2 fold change of <−0.5 or >1 were considered differentially expressed. Biological significance of differentially expressed genes can be identified at the pathway level using various gene sets, including but not limited to MSigDB (Molecular Signatures Database, RRID:SCR_016863) hallmark gene sets and KEGG (RRID:SCR_012773) gene sets.

4. Neoantigen-Presentation Prediction

The set of transcriptomic metrics can include a quantitative or categorical metric that represents one or more characteristics of each of one or more HLA proteins for which a loss of cell-surface presentation is detected. In particular, the transcriptomic metric can correspond to patient specific tumor alterations that could interfere with neoantigen presentation, including HLA mutations, HLA loss of heterozygosity, and beta-2-microglobulin mutations.

The neoantigen-presentation prediction metric can be generated by identifying candidate neoantigens generated using tumor-specific genomic events (single-nucleotide variants, indels, and fusions) that were verified using the transcriptomic data. All candidate peptides can be scored using a neoantigen-presentation-prediction machine-learning model for predicting MHC class I presentation, which can be trained using large scale immunopeptidome datasets. The trained neoantigen-presentation-prediction machine-learning model can use data corresponding to each of the candidate peptides to generate an output that predicts whether the candidate peptide will be presented and expressed on the cell surface. Based on the output of the machine-learning model, a neoantigen burden score can be calculated using a subset of candidate peptides that pass a confidence threshold. To calculate the composite biomarker score, the neoantigen burden score can be adjusted to account for subject-specific tumor alterations which may impair neoantigen presentation, including alterations to the MHC complex and antigen presentation machine and HLA loss of heterozygosity.

C. Genomic Metrics Derived from Genomic Data

The immunogenomics-analysis system can process the genomic data to generate a set of genomic metrics. Each of the set of genomic metrics can represent one or more characteristics corresponding to a corresponding DNA sequence the one or more DNA sequences. In some instances, the set of genomic metrics include: (i) a quantitative or categorical metric that represents one or more characteristics for each of one or more somatic mutations in the one or more DNA sequences; (ii) a categorical metric that indicates whether a loss of heterozygosity has occurred in at least one human leukocyte antigen (HLA) gene of the biological sample; and (iii) a quantitative or categorical metric that represents a predicted tumor mutational burden. With respect to the HLA loss of heterozygosity, the corresponding categorical metric can be generated by applying the genomic data to an HLA-deletion-identification machine-learning model.

1. Single-Nucleotide Variants and Indels

The set of genomic metrics can include a quantitative or categorical metric that represents one or more characteristics for each of one or more somatic mutations in the one or more DNA sequences. The one or more somatic mutations can include single-nucleotide variants, insertion/deletion polymorphisms, copy number alterations, and fusions in one or more nucleic acid molecules of the DNA sequences. In some instances, quality metrics can be generated for each identified mutation in the DNA sequences, including number of mutations, a ratio of transition to transversion, variant-level concordance, etc. For example, the genomic data can be processed using a quality score recalibration module, which can stratify single-nucleotide variants by their likelihood of representing false positive calls. In some instances, sequence alignment information of the genomic data can be processed such that miscalled variants can be corrected. Additionally or alternatively, somatic single-nucleotide variants and indel calls can be combined and analyzed through a tested set of filters based on 1) alignment metrics, such as sequence coverage and read quality, 2) positional features, such as proximity to a gap region, and 3) likelihood of presence in normal tissue.

2. Allele-Specific HLA Loss of Heterozygosity

The set of genomic metrics can also include a categorical metric that indicates whether a loss of heterozygosity has occurred in at least one HLA gene of the biological sample. HLA loss of heterozygosity can be detected using a HLA-deletion-identification machine-learning model, as HLA loss of heterozygosity can impact neoantigen presentation. HLA loss of heterozygosity can be considered as an acquired resistance mechanism that facilitates immune escape by reducing capacity for presentation of tumor neoantigens to the immune system. As the process of HLA loss is governed by selective pressures within the tumor microenvironment, particularly at later stages of tumor evolution, it was hypothesized that within the cohort of late-stage melanoma subjects allele-specific HLA loss of heterozygosity could contribute to reduced therapeutic response despite apparent elevated neoantigen burden.

To generate the above genomic metric, the biological sample can processed using the following steps: 1) all tumor and normal reads were mapped to the subject's allele-specific HLA; 2) homologous alleles were aligned to find all patient-specific mismatch positions; and 3) normalized b-allele frequencies and allele-specific coverage ratios were calculated at each mismatch position. For each gene, allele-specific features were input into the HLA-deletion-identification machine-learning model to predict loss of heterozygosity, including normalized b-allele frequencies and allele-specific mismatched positions, tumor purity, and tumor ploidy.

3. Mutational Burden

The set of genomic metrics can include a quantitative or categorical metric that represents a predicted tumor mutational burden. The tumor mutational burden can refer to the total number of mutations (changes) found in the DNA of cancer cells. Knowing the tumor mutational burden may help plan the best treatment, and the tumor mutational burden has been identified as a potential biomarker for immune checkpoint blockade response.

D. Generating the Composite Biomarker Score

The immunogenomics-analysis system generates a composite biomarker score derived from the set of genomic metrics and the set of transcriptomic metrics and determines, based on the composite biomarker score, a predicted level of responsiveness of the subject to a particular type of an immunotherapy treatment. For example, the composite biomarker score can be generated by using the transcriptomic metric corresponding to a neoantigen burden score, which can be adjusted based on the predicted tumor mutational burden identified from the genomic data. The composite biomarker score can thus account for impairment to neoantigen presentation and other established resistance markers. Integrating antigen presentation into the composite biomarker score may strengthen prediction levels associated with immune checkpoint blockade response.

While elevated measures of neoantigen burden may be predictive of which subjects will benefit from immunotherapy, the composite biomarker score can be derived based on genomic and transcriptomic metrics corresponding to additional resistance mechanisms arising from genetic variation in the antigen presentation machinery, both at a germline as well as somatic level. These additional resistance mechanisms can further modulate immune response by diminishing capacity for neoantigen presentation. Thus, the composite biomarker can use the metric corresponding to neoantigen burden as a biomarker, but can further include genomic and transcriptomic metrics corresponding to additional data derived subsequent processing steps and longitudinal treatments, as well as RNA expression levels.

In some instances, the composite biomarker score corresponds to an neoantigen burden score that is adjusted to account for subject specific tumor alterations that could further interfere with neoantigen presentation, including HLA mutations, HLA loss of heterozygosity, and beta-2-microglobulin mutations. As a result, analysis of subjects using the composite biomarker score can result in improved prediction of therapy outcome, when compared to neoantigen and tumor mutational burden individually. A composite biomarker approach that models both biological mechanisms and impairment of neoantigen presentation can serve as a stronger biomarker for immune checkpoint blockade therapy than many of the current biomarkers built around simpler biological models of tumor immune response. Unlike tumor mutational burden-based approaches, the composite biomarker score can be generated by modeling broader mechanisms of neoantigen presentation.

Additionally or alternatively, a subset of somatic mutations associated with reduced response to immunotherapy (e.g., HLA class I and B2M mutations, loss of heterozygosity in HLA class I genes) are weighted to adjust the composite biomarker score. By accounting for these escape mechanisms, the composite biomarker score can capture a fuller representation of tumor antigen presentation to the immune system to increase the predictive strength of this biomarker. The above approach can produce more accurate results when applied to one or more specific types of cancers, such as non-small-cell lung carcinoma and squamous cell carcinoma of the head and neck subject cohorts, since HLA loss of heterozygosity was identified as a prevalent escape mechanism that affects cancer progression for those types. For example, tumor data revealed allele-specific expression loss at frequencies above 45% in head and neck, lung adenocarcinoma, pancreatic and prostate cancers. HLA loss of heterozygosity, combined with the prevalence of somatic mutations in class I HLA genes can be captured by the composite biomarker score to identify damaging events to antigen presenting machinery.

Thus, the composite biomarker score can integrate a broad set of biological features across multiple dimensions: exome and transcriptome, tumor and immune, response and resistance. The composite biomarker score can then be used for predicting immune checkpoint blockade response that reflect the biological mechanisms driving response and resistance to immunotherapies.

E. Treatment Selection

The composite biomarker score can serve as a strong predictor for immune checkpoint blockade therapy response. As shown in the figures, the composite biomarker score achieved greater separation of immune checkpoint blockade therapy responders and non-responders than tumor mutational burden and other single analyte/gene, and expression signatures examined in the discovery cohort. The value of the composite biomarker score for predicting responsiveness to particular immunotherapies was further demonstrated by confirming these findings in a large independent validation cohort.

The composite biomarker score can further demonstrate that neoantigens can guide immune response, promoting clinical response to immunotherapy. While only weak association was observed between response and tumor mutational burden, stronger association between neoantigen burden and subject response was apparent. It has been suggested that this finding may be attributed to confounding effects of the distribution of melanoma subtypes within patient cohorts in various clinical studies, which negatively impact the predictive power of tumor mutational burden. However, such issues involving the cohorts did not appear to affect neoantigen burden. It is possible that the increased robustness of neoantigen burden as a biomarker was achieved through the inclusion of additional data from subsequent processing steps, as well as RNA expression levels, as this measure has been found to correlate with protein representation in the MHC-bound peptide repertoire.

In some instances, additional factors influencing subject response are identified outside of neoantigen burden. As an illustrative example, within the discovery cohort, non-responding outlier with the highest observed composite biomarker score also includes a high impact, nonsense PD-1 mutation, which can be interpreted as likely preventing response to anti-PD1 therapy. The outlier, non-responding subject in the validation cohort with high composite biomarker score corresponds to a subject with metastatic desmoplastic melanoma, which is associated with high levels of mutational burden and distinct clinicopathologic and genetic features compared to typical cutaneous melanomas. Thus, using clinical response data with the composite biomarker score can identify a level of heterogeneity of subject response to immunotherapies. Further, the combination of clinical response data with the composite biomarker score can identify subsets of malignancies vulnerable to specific therapy combinations. Finally, the combination of clinical response data with the composite biomarker score can identify other mechanisms of therapy resistance or response that extend beyond neoantigen presentation.

The composite biomarker score can thus be used to determine a treatment method to prevent, arrest, reverse, or ammeliorate a disease. The disease may be a cancer. The composite biomarker score can indicate a predicted level of responsiveness of the subject. Accordingly, the composite biomarker score can be outputted as a be report that identifies, based on the predicted level of responsiveness of the subject to the particular treatment: (i) a treatment recommendation of the particular treatment; (ii) a recommendation to administer the particular treatment to the human subject; and/or (iii) a recommendation to not administer the particular treatment to the human subject. In some embodiments, the recommended treatment is administered to the human subject.

Non-limiting examples of cancers include: acute lymphoblastic leukemia, acute myeloid leukemia, adrenocortical carcinoma, AIDS-related cancers, AIDS-related lymphoma, anal cancer, appendix cancer, trocytomas, neuroblastoma, basal cell carcinoma, bile duct cancer, bladder cancer, bone cancers, brain tumors, such as cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas, Burkitt lymphoma, carcinoma of unknown primary origin, central nervous system lymphoma, cerebellar astrocytoma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing s sarcoma, germ cell tumors, gallbladder cancer, gastric cancer, gastrointestinal carcinoid tumor, gastrointestinal stromal tumor, gliomas, hairy cell leukemia, head and neck cancer, heart cancer, hepatocellular (liver) cancer, Hodgkin lymphoma, Hypopharyngeal cancer, intraocular melanoma, islet cell carcinoma, Kaposi sarcoma, kidney cancer, laryngeal cancer, lip and oral cavity cancer, liposarcoma, liver cancer, lung cancers, such as non-small cell and small cell lung cancer, lymphomas, leukemias, macroglobulinem malignant fibrous histiocytoma of bone/osteosarcoma, medulloblastoma, melanomas, mesothelioma, metastatic squamous neck cancer with occult prima mouth cancer, multiple endocrine neoplasia syndrome, myelodysplastic syndromes, myeloid leukemia, nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer, ovarian germ cell tumor, pancreatic cancer, pancreatic cancer islet cell, paranasal sinus and nasal cavity cancer, parathyroid cancer, penile cancer, pharyngeal cancer, pheochromocytoma, pineal astrocytoma, pineal germinoma, pituitary adenoma, pleuropulmonary blastoma, plasma cell neoplasia, primary central nervous system lymphoma, prostate cancer, rectal cancer, renal cell carcinoma, renal pelvis and ureter transitional cell cancer, retinoblastoma, rhabdomyosarcoma, salivary gland cancer, sarcomas, skin cancers, skin carcinoma merkel cell, small intestine cancer, soft tissue sarcoma, squamous cell carcinoma, stomach cancer, T-cell lymphoma, throat cancer, thymoma, thymic carcinoma, thyroid cancer, trophoblastic tumor (gestational), cancers of unknown primary site, urethral cancer, uterine sarcoma, vaginal cancer, vulvar cancer, Waldenstr6m macroglobulinem and Wilms tumor. Examples of diseases or conditions in which an integrative, composite biomarker can be employed include hematological malignancies, solid tumor malignancies, metastatic cancer, and benign tumors.

A plurality of subjects afflicted with cancers can benefit from the use of an integrative, composite biomarker. Subjects can be humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. A subject can be of any age. Subjects can be, for example, elderly adults, adults, adolescents, pre-adolescents, children, toddlers, infants.

Patient health or treatment options may be assessed by providing a bodily fluid or tissue sample from a subject; collecting a genomic and proteomic profile from the bodily fluid or tissue sample and comparing the genomic and proteomic profiles to at least one reference profile to assess the health of the subject. The reference profile may profile at least one of: one or more disease, injury or disorder. The reference profile may be established from the genomic or proteomic profile collected from subjects with the same disease, from a healthy population, or both. The method may comprise monitoring by repeatedly comparing, over time, the genomic or proteomic profile to the reference profile. Aspects of the present disclosure may comprise statistically analyzing differences between a tumor profile and reference profile to identify at least one biomarker. Biomarkers or a group of biomarkers having a significance level of less than 95%, 97% 98% or 99% may be rejected.

In some aspects, the present disclosure may provide a method of adaptive immunotherapy for the treatment of cancer in a subject comprising administering a first course of a first immunotherapy compound to the subject; acquiring comprehensive tumor and immune related molecular information relating to additional emerging and investigational biomarkers such as neoantigen burden, HLA genotype diversity, I A loss of heterozygosity, immune repertoire profiles, immuno-cellular deconvolution, oncoviruses, and more, wherein the second course of immunotherapy comprises a second immunotherapy compound if the tumor and immune related molecular profile is indicative of an insufficient response to the first immunotherapy compound; or a second course of the first immunotherapy compound if the tumor and immune related molecular profile is not indicative of an insufficient response to the first immunotherapy compound. One or more biological samples acquired after administering a first dose of a first course of a first immunotherapy compound may be acquired on the same day that a subsequent dose of the first course of a first immunotherapy compound may be administered.

Treatment, testing, or analysis may be provided to the subject before clinical onset of disease. Treatment, testing, or analysis may be provided to the subject after clinical onset of disease. Treatment, testing, or analysis may be provided to the subject after Iday, Iweek, 6 months, 12 months, or 2 years after clinical onset of the disease. Treatment, testing, or analysis may be provided to the subject for more than Iday, Iweek, Imonth, 6 months, 12 months, 2 years or more after clinical onset of disease. Treatment, testing, or analysis may be provided to the subject for less than Iday, I week, Imonth, 6 months, 12 months, or 2 years after clinical onset of the disease. Treatment, testing, or analysis may also include treating, testing, or analyzing a human in a clinical trial.

### VI. Experimental Results for Generating a Composite Biomarker Score

To demonstrate the effectiveness of the composite biomarker score in predicting immune-system response to immunotherapies, the following experiment was conducted. Paired pretreatment formalin-fixed paraffin-embedded tumor and normal blood samples was collected and profiled to produce comprehensive tumor mutation information, gene expression quantification, neoantigen characterization, HLA (typing, mutation, and loss of heterozygosity), TCR repertoire profiling, and tumor microenvironment profiling. These data were then comparted together with clinical outcome, at which the composite neoantigen score was computed for each subject along with additional biomarkers such as tumor mutational burden.

A. Cohort Population

1. Discovery Cohort

With respect to the study population, 51 subjects with unresectable, stage III/IV melanoma who underwent treatment were enrolled retrospectively without randomization or blinding. Subjects were treated with either nivolumab (480 mg IV every 4 weeks or 240 mg IV every 2 weeks), a combination of nivolumab and ipilimumab (1 mg/kg IV and 3 mg/kg IV, respectively, every 3 weeks), or pembrolizumab (200 mg IV every 3 weeks). Solid tumor and blood samples were collected within three months prior to treatment start. Computed tomographic scans were performed 10-12 weeks after treatment start, with follow-up scans every three months. Responders were defined as complete response (CR) or partial response (PR). Non-responders were defined as stable disease (SD) or progressive disease (PD).

2. Validation Cohort

Replication of the predicted results was conducted using publicly available NGS data collected from advanced melanoma subjects who underwent immune checkpoint blockade therapy. Whole exome and RNA sequencing data from this study were obtained from dbGaP (NCBI database of Genotypes and Phenotypes, RRID:SCR_002709) (study accession: phs000452.v3.p1). Subjects with mixed responses to therapy (n=2) and low purity tumors (n=7) were excluded from the analysis, leaving (n=110) evaluable subjects for validation. Clinical characteristics for the validation cohort are provided in the original study.

3. Statistical Analysis for Clinical Data

With respect to generating clinical data, the Kaplan—Meier method was used to estimate progression-free survival (PFS) and overall survival (OS). Objective response rate was reported as proportion along with Clopper—Pearson exact CIs. Fisher's exact test and chi-square test were used to test for associations between groups, and categorical variables. When considering the variance between more than two groups, the Kruskal-Wallis H test was used. The Wilcoxon Mann—Whitney rank sum test (MWW) was used for numeric pairwise comparisons. Benjamini-Hochberg correction was used to adjust P values as listed. The Kolmogorov-Smirnov (KS) statistic was used for RNA pathway analyses. Correlations between continuous variables were determined using Kendall's tau. Predictive models were generated using logistic regression, and AUROC used to determine ability to differentiate between response and non-response according to published methods (28). All tests were two-sided; FDR values of <0.1 for pathway analyses, and P-values of <0.05 for all other tests were considered statistically significant. The following table provides

B. Cohort Clinical Data

1. Clinical Characteristics Corresponding to Samples of the Discovery Cohort

For the 51 unresectable melanoma subjects in the cohort treated with immune checkpoint blockade, median follow-up time period corresponding to the cohort was 24 months after treatment, with 33 out of 51 subjects (50%, 95% Clopper—Pearson confidence interval of 50-78%) presenting an objective response at first evaluation by Response Evaluation Criteria in Solid Tumors (RECIST) 1.1. Within the clinical cohort, tumors originated in the head and neck region (31%), trunk (31%), extremities (25%), acral areas (6%), mucosa (4%), and 2% from occult regions. In addition to these data, sex, age and other subject-specific demographics information is presented. The following table provides a summary of various characteristics associated with subject of the clinical cohort:

TABLE 1
Discovery Cohort Characteristics

| n | Responder (33) | Non-responder (18) | P-value |
|---|---|---|---|
| Age at treatment | 68(61-81) | 66.5(56.5-76) | 0.4536 |
| Disease origin | | | 0.9901 |
| Acral | 2(6.1%) | 1(5.6%) | |
| Extremity | 8(24.2%) | 5(27.8%) | |
| Head neck | 11(33.3%) | 5(27.8%) | 0.926 |
| Mucosal | 1(3%) | 1(5.6%) | |
| Trunk | 1(3%) | 0%) | |
| PD1 therapy | | | |
| Nivolumab | 10(30.3%) | 6(33.3%) | 0.8976 |
| Nivolumab (in | 8(24.2%) | 6(33.3%) | 0.7137 |
| combination with | | | |
| ipilimumab) | | | |
| Pembrolizumab | 5(15.2%) | 2(11.1%) | |
| Sex | | | |
| Female | 1(3%) | (0%) | |
| Male | 19(57.6%) | 10(55.6%) | 0.7513 |
| Stage at treatment | | | |
| Unresectable Ill | 9(27.3%) | 6(33.3%) | 0.8947 |
| M1a | 24(72.7%) | 12(66.7%) | 0.8947 |
| M1b | 1(3%) | 1(5.6%) | 0.2068 |
| M1c | 5(15.2%) | 0%) | 0.2127 |

As shown in Table 1, there was no statistically significant difference in objective response rates between sites of disease origin. Further, 11 subjects (22%) had progressed following prior treatment with a checkpoint inhibitor, whereas 40 (78%) were naive to immune checkpoint blockade. Subjects were administered either pembrolizumab (n=29, 57%), nivolumab (n=15, 29%), or a combination of nivolumab and ipilimumab (n=7, 14%).

2. Genomic Data Corresponding to Samples of the Discovery Cohort

Mutations associated with responding and non-responding tumors were investigated, revealing no significant single-gene predictors of response following multiple hypothesis correction (subject-level mutation data). The following table lists log 2 and p-values that provides comparison data between responders and non-responders for each identified gene in the clinical cohort:

TABLE 2

| Gene | log2 fold chang | adj p value |
|---|---|---|
| C10RF204 | −1.61163 | 0.018108 |
| CRHRl-ITl | −1.07242 | 0.03795 |
| CTD-2201118 | −1.60283 | 0.018993 |
| CXORF22 | −2.36416 | 0.026933 |
| DLL3 | 3.281187 | 0.000492 |
| DRD4 | −1.47017 | 0.002091 |
| JMJD6 | 0.581033 | 0.018885 |
| IL12RB2 | 1.805639 | 0.010296 |
| NEFH | 2.332242 | 0.040698 |
| KCNKlO | −1.65424 | 0.043177 |
| VSIGl | −1.68421 | 0.009372 |
| GLRA2 | 4.046629 | 0.002194 |
| SALLl | −2.69747 | 0.009372 |
| CEMIP | −1.82324 | 0.00129 |
| RSPH6A | −1.501 | 0.030373 |
| LRP2BP | −0.7731 | 0.025208 |
| MGP | 2.455289 | 0.003771 |
| FGF12 | −2.42734 | 0.000436 |
| APC2 | −1.94058 | 0.009374 |
| GBPl | 1.433309 | 0.046076 |
| TNNT2 | −2.13612 | 0.018993 |
| IRFl | 1.215632 | 0.026263 |
| AMOT | 1.175707 | 0.00657 |
| RASLllB | −1.60126 | 0.034151 |
| FCHOl | −1.19714 | 0.030651 |
| 1DO1 | 1.939955 | 0.032344 |
| GUCY2D | −1.26331 | 0.048385 |
| ZNF767P | −1.72851 | 0.030087 |
| CATSPERB | −1.81093 | 0.021066 |
| TIC9 | −1.57921 | 0.024555 |
| CRBl | 2.09183 | 0.043581 |
| BAAT | 3.01605 | 0.015062 |
| Pl15 | 2.534018 | 0.016874 |
| ARHGAP20 | 1.468957 | 0.037466 |
| CXCL9 | 2.198974 | 0.002194 |
| LMANlL | −1.63068 | 0.030651 |
| MYLK3 | −1.64958 | 0.02731 |
| ZNF385B | −1.99676 | 0.02731 |
| STXBP5L | −3.24565 | 0.000812 |
| RNF175 | 2.592116 | 0.015062 |
| CRB2 | −1.98509 | 0.016586 |
| GLB1L2 | −1.76366 | 0.035468 |
| TAOK2 | −0.84718 | 0.048927 |
| GPM6A | −3.0352 | 0.001369 |
| ASTNl | −2.0683 | 0.016683 |
| GDPDl | 1.373747 | 0.003771 |
| CDH12 | −2.8287 | 0.009372 |
| EPHBl | −1.86788 | 0.010296 |
| PPMlJ | −1.34045 | 0.006808 |
| PAQR6 | −0.97253 | 0.042537 |
| SHANKl | −1.607 | 0.030373 |
| FGFll | −1.54424 | 0.014214 |
| ANKRDSS | −1.79874 | 0.037442 |
| SMC02 | −1.66042 | 0.030505 |
| ELFN2 | 2.718762 | 0.009374 |
| CXCLlO | 1.87592 | 0.00657 |
| CXCLll | 2.002743 | 0.005084 |
| KRT86 | 3.173565 | 0.003247 |
| KRT72 | −3.6212 | 0.000436 |
| ZNF540 | −1.09009 | 0.0358 |
| AQP4 | −4.01248 | 0.002491 |
| MBOATl | 0.699832 | 0.032344 |
| SUSD5 | 2.027636 | 0.030651 |
| SLC16A13 | −0.93716 | 0.048528 |
| DOK7 | −1.8985 | 0.026074 |
| ZNF575 | −0.68771 | 0.036673 |
| DSCAMLl | −2.13465 | 0.024377 |
| CITED4 | −2.07691 | 0.005867 |
| CSNKlAlL | −1.20759 | 0.037437 |
| SYNE4 | −1.24594 | 0.034861 |
| SAGEl | 4.264828 | 0.009372 |
| IZUMOl | −1.307 | 0.027117 |
| ZDHHC23 | −1.45714 | 0.032344 |
| LCN12 | −1.58892 | 0.005125 |
| KRT73 | −3.12158 | 0.001387 |
| NANOS3 | −1.66023 | 0.030373 |
| ANKRD19P | −2.57316 | 0.000492 |
| SLC38A3 | −2.25653 | 0.036738 |
| HBA2 | −1.70692 | 0.017059 |
| ZNF560 | −2.87698 | 0.009372 |
| CACNAl E | 1.832572 | 0.041874 |
| SNORA60 | −1.39145 | 0.015062 |
| HSPAlL | −1.07768 | 0.018366 |
| APOM | −1.02226 | 0.026263 |
| KRT81 | 3.180689 | 0.009372 |
| IGLL3P | −3.76388 | 0.002117 |
| EGFEMlP | 2.988888 | 0.002117 |
| HBAl | −1.65165 | 0.021066 |
| UBD | 1.715684 | 0.021069 |
| SNORA7B | −1.32328 | 0.027766 |
| SNORA68 | −1.46923 | 0.041874 |
| ARHGEF35 | −2.07861 | 0.02636 |
| PPMlN | −1.00687 | 0.048927 |
| LTB4R2 | −1.42895 | 0.037079 |
| TIC3Pl | −0.89718 | 0.030087 |
| SNORA77 | −1.30542 | 0.035468 |
| LINC00115 | −0.71744 | 0.048482 |
| TMEM238 | −1.58165 | 0.02258 |
| UGT1A5 | −2.27141 | 0.003063 |
| UGT1A3 | −2.53733 | 0.000679 |
| UPK3B | −1.23583 | 0.018108 |
| LCE3C | −5.31202 | 0.026263 |
| HBB | −1.97396 | 0.002194 |
| ANP32C | −1.25361 | 0.015062 |
| TGFBR3L | −1.68206 | 0.018807 |
| MIR5690 | −1.82684 | 0.009372 |
| C2orf15 | −0.98529 | 0.04459 |
| NEFL | −2.51474 | 0.015062 |
| FLJ39080 | 2.796046 | 0.021066 |
| LMTK3 | −1.25002 | 0.012716 |
| LOC100131691 | −1.45461 | 0.042537 |
| LOC100289580 | −1.29238 | 0.027375 |
| LOC100506388 | −1.53284 | 0.030651 |
| LOC100652768 | −1.05443 | 0.024471 |
| LOC100653133 | −1.9145 | 0.018993 |
| LOC101927372 | −1.30701 | 0.041557 |
| LOC101928179 | −1.66223 | 0.030505 |
| LOC101928457 | −5.60655 | 0.000436 |
| LOC101928577 | −1.3549 | 0.009372 |
| LOC399900 | −0.85131 | 0.043177 |
| TCLlA | −1.87922 | 0.032344 |

3. Immune Pathway Data Corresponding to Subjects of the Cohort

Next, genetically disrupted pathways corresponding to the clinical data were determined. The most frequently disrupted pathways included RTK-RAS and WNT pathways (disrupted in 73% and 51% of our cohort, respectively). Mutations were detected throughout the RTK-RAS pathway. Numerous RTKs were mutated including ROS1 and ERBB4, RAS family genes including NRAS, BRAF, and MAPK1 and 2.

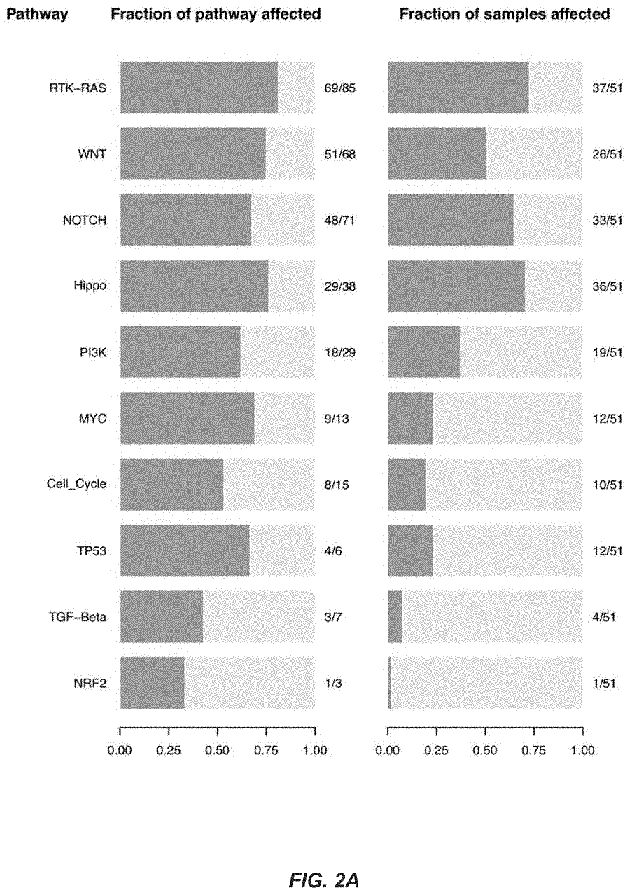

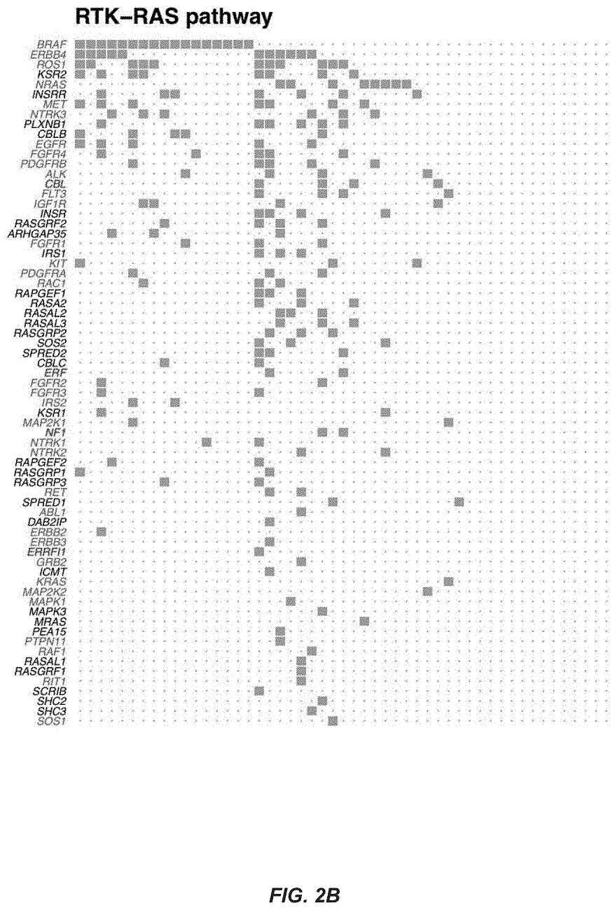

FIGS. 2A-B show statistical data corresponding to oncogenic changes in genomic and transcriptomic data corresponding to subjects of a clinical cohort. FIG. 2A shows mutations in known oncogenic pathways in late stage melanoma subjects. In FIG. 2A, fraction of pathway affected denotes the number of genes mutated within the pathway (n=51 samples included in this analysis). FIG. 2B shows visualization of mutations occurring within the RTK-RAS pathway. Tumor suppressor genes are listed in red, and oncogenes are shown in blue. Dots represent absence of mutation within the specified gene. Each column represents a tumor, with green blocks representing variants within a given gene.

C. Transcriptomic Metrics

1. Differential Gene Expressions

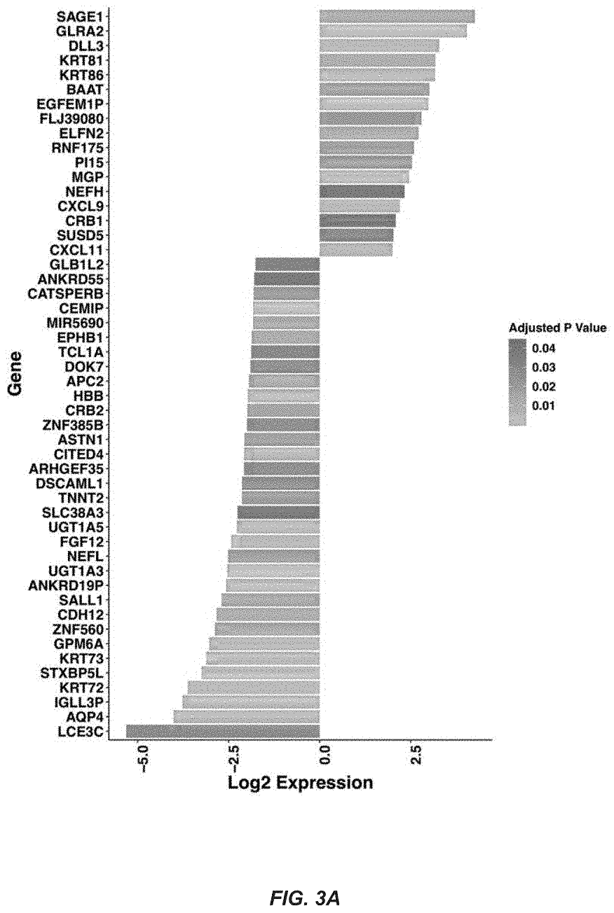

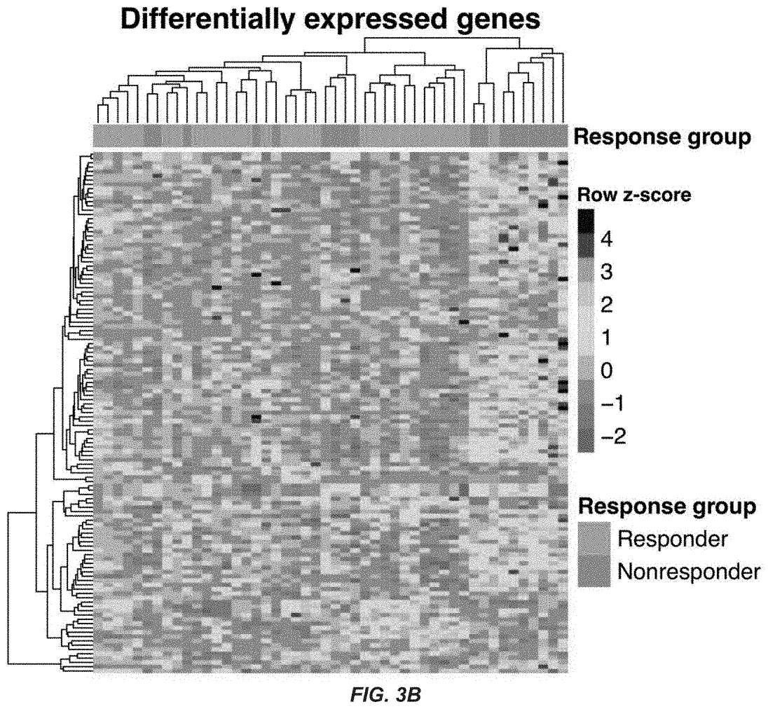

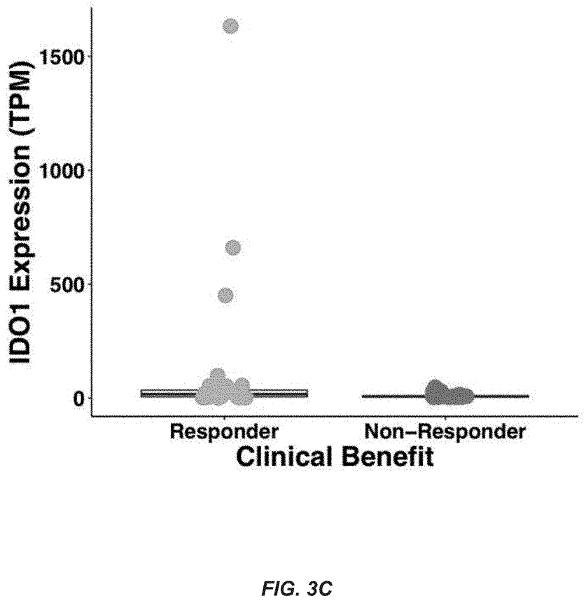

Transcriptomic data was generated for each subject in the discovery cohort. From the transcriptomic data, various transcriptomic metrics were generated. For example, 121 differentially expressed genes were identified in responding subjects (n=48 evaluable subjects; adjusted P-value ≤0.05, log fold change >2 or <−0.5). FIGS. 3A-C show statistical data corresponding to transcriptomic metrics that identify differentially expressed genes that are associated with immune-system response. FIG. 3A shows 50 genes with highest levels differential expressions in the cohort, in which fold change has been provided to compare responding subjects to non-responding subjects. FIG. 3A further shows Benjamini-Hochberg corrected P values below 0.05 for each gene of a corresponding set of 48 genes. Although all are not shown in FIG. 3A, enrichment was observed in 29 of these genes, while reduced expression was observed in 92 genes. To illustrate, FIG. 3B shows a heatmap of differentially expressed genes for each subject of the clinical cohort. In FIG. 3B, each column represents a subject, and each row represents a gene.

Among the most strongly upregulated genes (log 2 fold change=3.28; FDR adjusted P=0.0005) included delta-like ligand 3 (DLL3), which is an inhibitory Notch ligand that exhibits high expression in small cell lung cancer and other tumors tissues. Because of its low cytoplasmic expression in normal tissue compared to elevated, homogeneous cell surface expression in tumors, the delta-like ligand 3 gene is currently under investigation as a possible therapeutic target. Additionally, four members of the keratin (KRT) family (KRT72, 73, 81, 86), which is a gene group identified to have extensive ties to cancer development, had altered expression levels when comparing responders and non-responders. Validation of gene expression analysis results for DLL3 and KRT family genes confirmed significance of DLL3 (MWW P=0.02), but not KRT72, 73, 81, 86 (MWW P=0.44, P=0.41, P=0.6 and P=0.17). Such difference in validation results can be possibly due to reduced sensitivity of determining differential expression in individual genes.

Though not significantly enriched at a cohort level, IDO1 expression was detected at very high levels in three subjects (median IDO1 TPM=10.36; outlier IDO1 TPM=1955, 661, and 451). To illustrate, FIG. 3C shows a set of box plots that compare IDO1 gene expression levels of responsive subjects and those of non-responsive subjects. The gene expression values were provided in units of Transcripts Per Kilobase Million. For the group of responsive subjects, three outlier subjects were identified. Although the IDO1 expression values did not appear to have a relationship with response to immunotherapies, these outliers with elevated levels of expression may indicate escape mechanisms that prevented complete response in corresponding subjects (n=48). For example, two of the subjects overexpressing IDO1 may have failed to achieve complete response to immunotherapies, possibly due to an IDO1-driven immunosuppressive environment.

2. Gene Enrichment Analysis

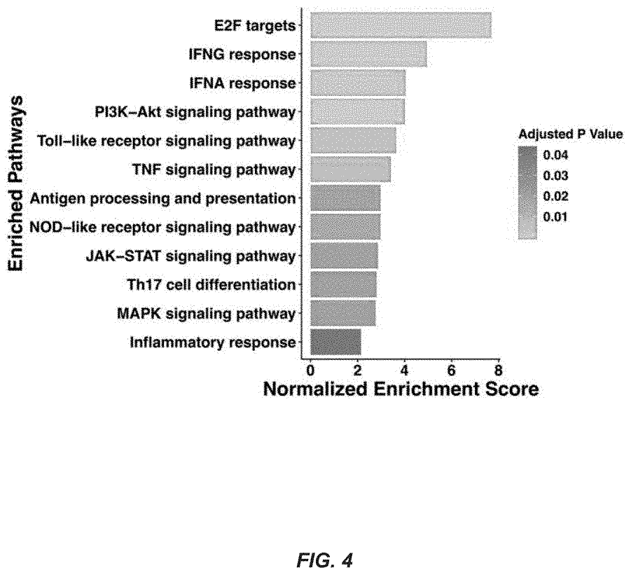

Next, gene set enrichment analysis was performed to identify differentially regulated pathways in the clinical cohorts. FIG. 4 shows statistical data corresponding to a normalized enrichment score for each differentially regulated immune pathway, in which the normalized enrichment scores are generated based on a gene-set enrichment analysis. In FIG. 4, significant enrichment of pathways related to immune function were identified among responsive subjects with up-regulated genes. Benjamini-Hochberg corrected P values below 0.05 are shown. Inflammatory signaling cascades were amongst the most highly enriched of those profiled (significance set as FDR<0.1). Activation of immune pathways likely have been resulted from other enriched pathways. For example, cellular differentiation of Th17 can be driven by: (i) the cytokine TGF-β, which induces RORγt in Th17 cells; and (ii) IL-6, which induces the Th17 lineage. The observed enrichment of Th17 may also be positively regulated by the observed increase in STAT3 signaling, which serves to promote Th17 differentiation.

3. Expression Levels of T-Cell Receptors

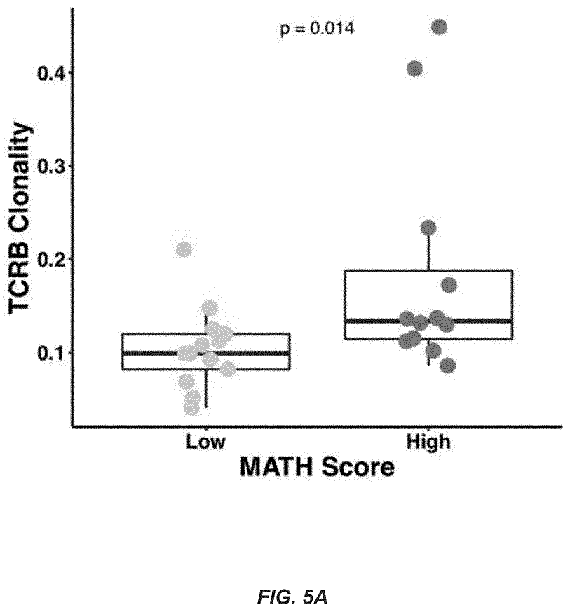

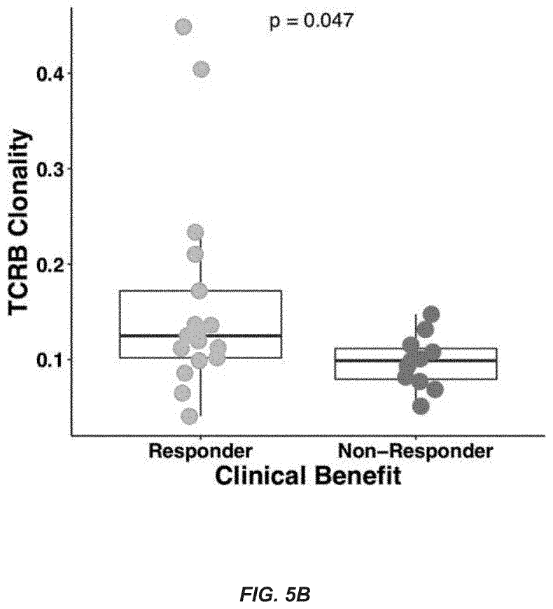

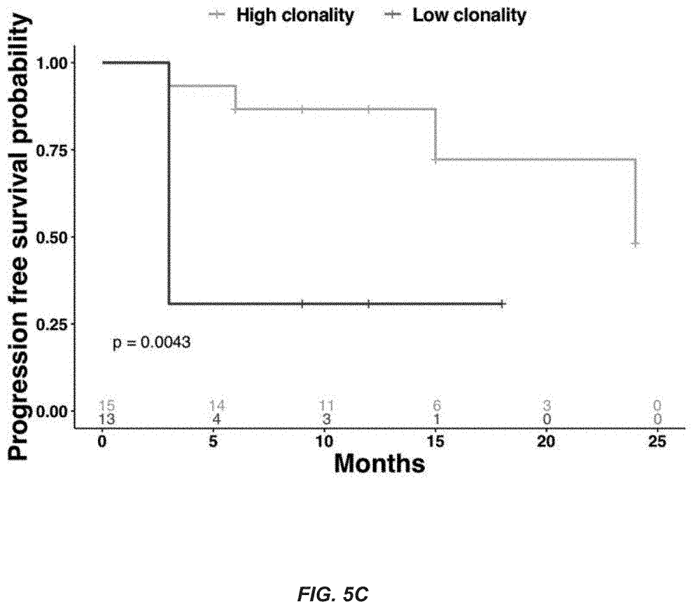

FIGS. 5A-C show statistical data corresponding to transcriptomic metrics that identify expression levels of T-cell receptors. The adaptive immune system can respond to a broad array of antigens due to its large repertoire of unique T-cell receptors (TCRs). The box plots in FIGS. 5A-B C cover the interquartile range from 25th percentile at their lower bound to the 75th percentile at their upper bound, with median indicated by a horizontal line. The upper whisker includes the largest value within 1.5× interquartile range above the 75th percentile. The lower whisker includes the smallest value within 1.5× interquartile range below 25th percentile. In order to characterize the pretreatment tumor-immune landscape, TCR-β repertoire diversity was identified in a subset of subjects in the cohort (n=28 subjects). Clonality was determined for the clonal abundance of all productive TCR-β sequences using 1-Pielou's evenness. As intra-tumoral heterogeneity is considered to be a determinant of immune response, mutant-allele tumor heterogeneity (MATH) scores, which indicate an estimated level of tumor heterogeneity, were compared with the identified TCR-β clonality. FIG. 5A shows a set of box plots that identify a comparison of TCR-β clonality between low and high mutant-allele tumor heterogeneity levels. As shown in FIG. 5A, a significant association (MWW, P=0.014) was identified between high tumor heterogeneity and clonal diversity of the TCR-β repertoire.

FIG. 5B shows a set of box plots that identify a comparison of TCR-β clonality between a first group of subjects identified as being responsive to immunotherapies and a second group of subjects identified as being non-responsive to the immunotherapies. As shown in FIG. 5B, TCR-β clonality is elevated in responding subjects, compared to nonresponders (n=28; MWW; P=0.047). Thus, TCR-β clonality can be considered to be significantly associated with therapy outcome. Further, FIG. 5C shows a line plot that identifies a comparison of progression-free survival probability between a first group identified to have high TCR-β clonality and a second group identified to have low TCR-β clonality. FIG. 5C shows that significantly longer progression free survival was observed in high clonality subjects when compared to those with low clonality (two-sided KM log-rank test, P=0.0043), in which high/low stratification was calculated independently for old/young populations (median cohort age used as cut point).

4. Immune Infiltrate Signatures

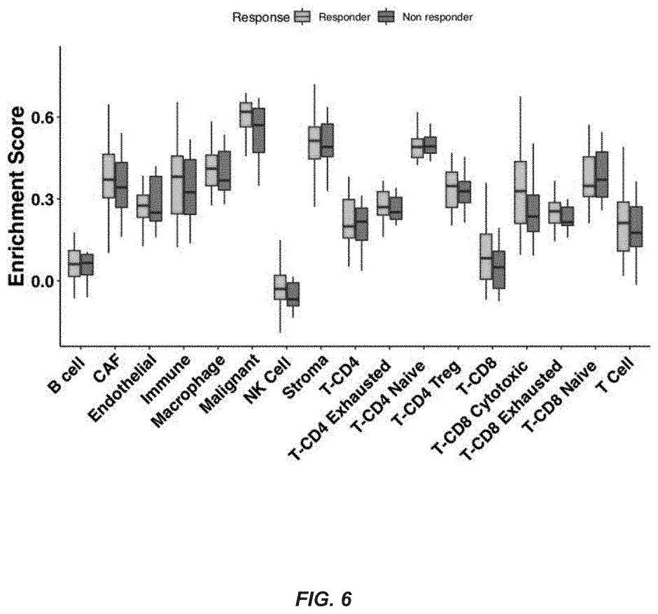

Characterization of immune and stromal cell populations within the tumor microenvironment (tumor microenvironment) in the cohort was implemented. The generated data were used to produce semi-quantitative immune infiltration scores. FIG. 6 shows a set of box plots that identify a comparison of enrichment scores between a first group of responsive subjects and a second group of non-responsive subjects. The comparison of enrichment scores was identified across various types of tumor infiltrating lymphocytes, including regulatory T-cell (TREG), natural killer cell (NK cell), and cancer associated fibroblast (CAF). As shown in FIG. 6, responding and non-responding subjects largely shared similar distributions of immune cell expression across various types. Thus, the gene expression levels of immune cell, in isolation, do not appear to be strong predictive indicator of responsiveness levels to immunotherapies. However, as described herein, the expression levels can be a contributing factor in generating the composite biomarker score that accurately predicts responsiveness to the immunotherapies.

5. Neoantigen Burden

A neoantigen-based biomarker approach achieves a strong correlation with response to immune checkpoint blockade. With respect to this particular exemplary experiment, two different neoantigen models were generated, such that their respective performance levels were compared. A first neoantigen model corresponded to a score based on neoantigen burden only, and a second neoantigen model corresponded to the first model that was extended to account for impairment to neoantigen presentation and other established resistance markers. The second neoantigen model thus corresponded to a model for generating the composite biomarker score.

To calculate the neoantigen burden score, features derived from exome- and transcriptomic data were used. Putative neoepitopes were predicted from single-nucleotide variants, indels, and fusions detected from both exome and transcriptome sequencing. To improve MHC class I neoantigen prediction, mass spectrometry-based peptide binding data from mono-allelic HLA transfected cell lines was generated. This data was used to train an improved machine learning algorithm which integrates HLA binding, proteasomal cleavage, and gene expression information to improve neoantigen prediction.

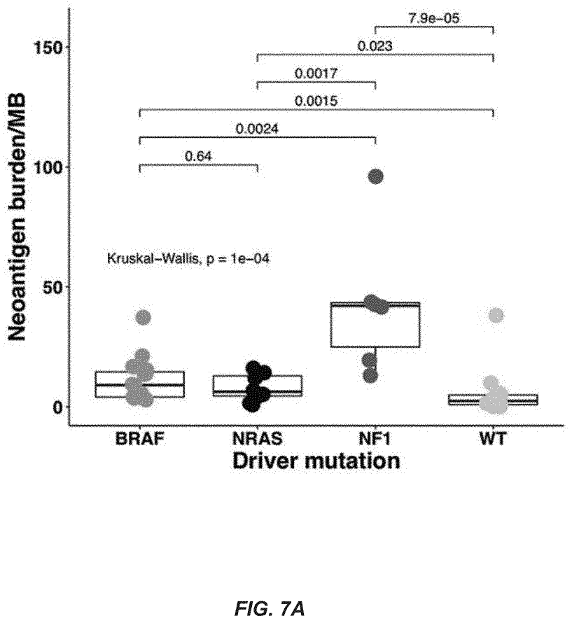

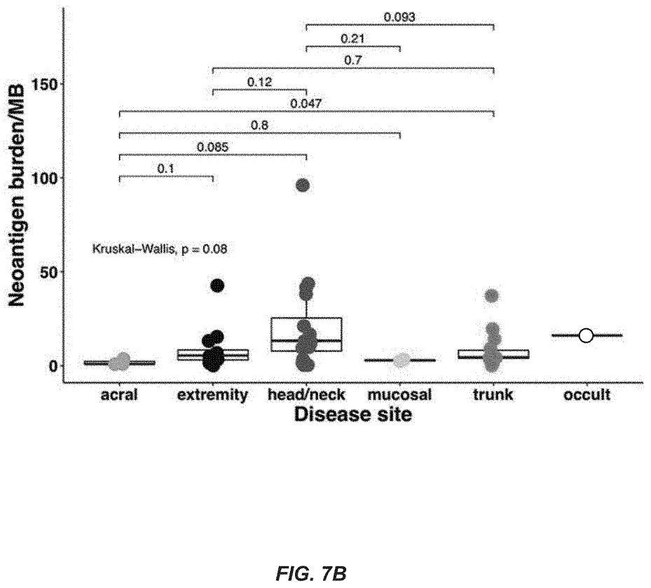

FIGS. 7A-B show statistical data corresponding to transcriptomic metrics that identify neoantigen burden across various genes and disease sites. FIG. 7A shows a set of box plots that identify neoantigen burden scores corresponding to driver mutations corresponding to BRAF, NRAS, NF1, and WT genes. FIG. 7A shows that neoantigen burden varied significantly between tumors harboring different driver mutations, revealing significant variation amongst subtypes (Kruskal—Wallis, P=1e-04).

In addition, FIG. 7B shows a set of box plots that identify neoantigen burden scores corresponding to various disease sites of melanoma, including acral, extremity, head/neck, mucosal, trunk, and occult regions. In FIG. 7B, a significant association across disease sites of origin was not detected (Kruskal—Wallis, P=0.08). Thus, neoantigen burden did not vary globally when comparing tumors arising from different sites of origin, although it can observed that post hoc comparison between acral and trunk melanomas did reveal significant variation (MWW; P=0.047).

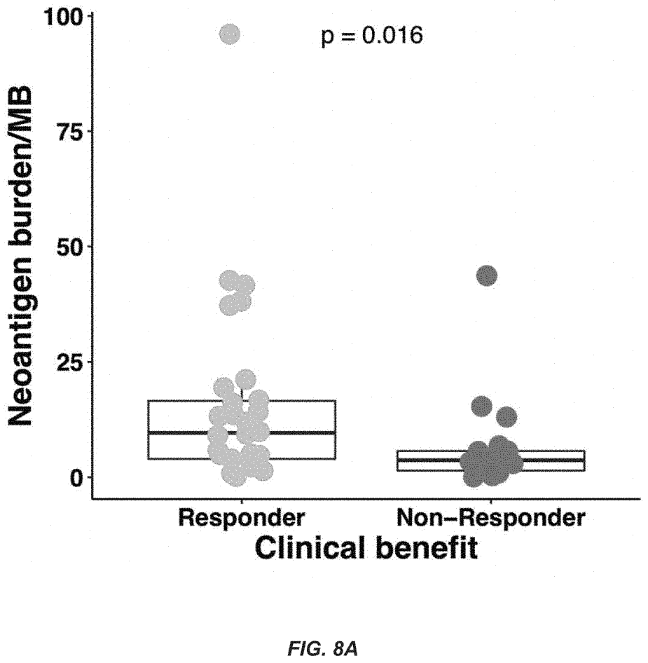

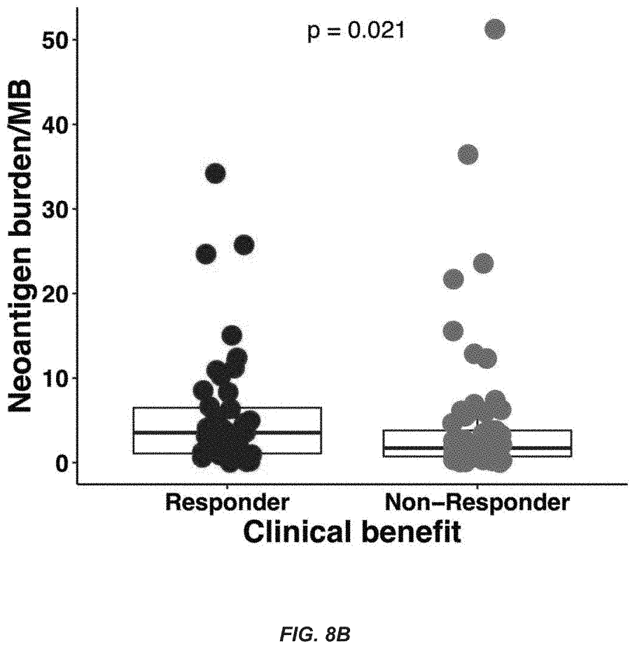

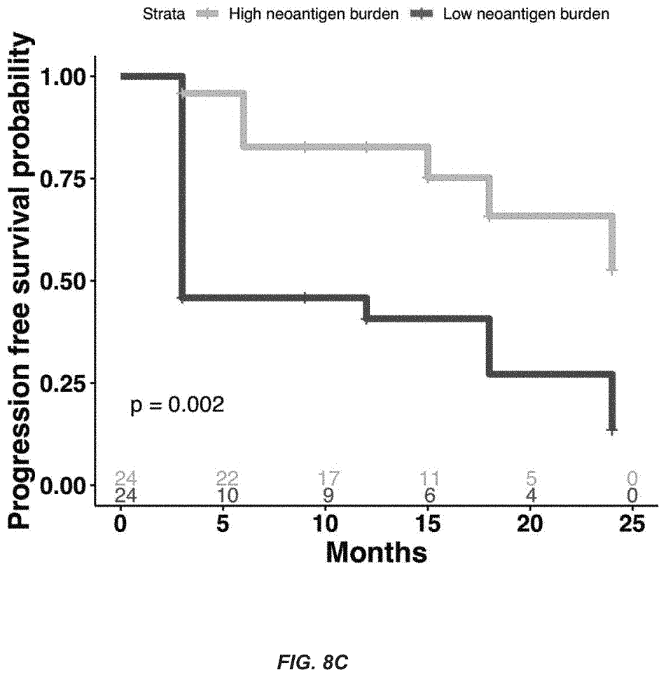

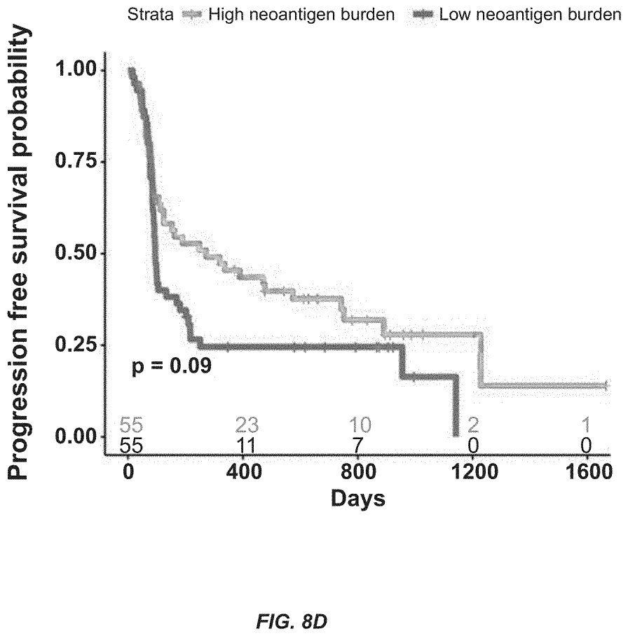

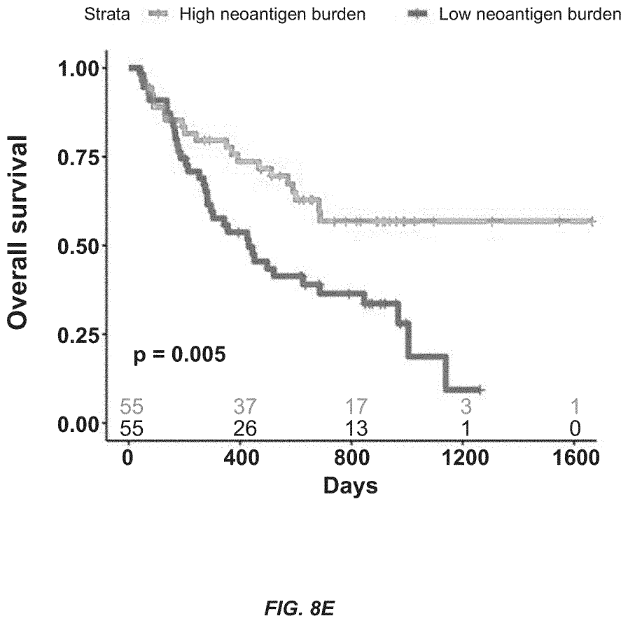

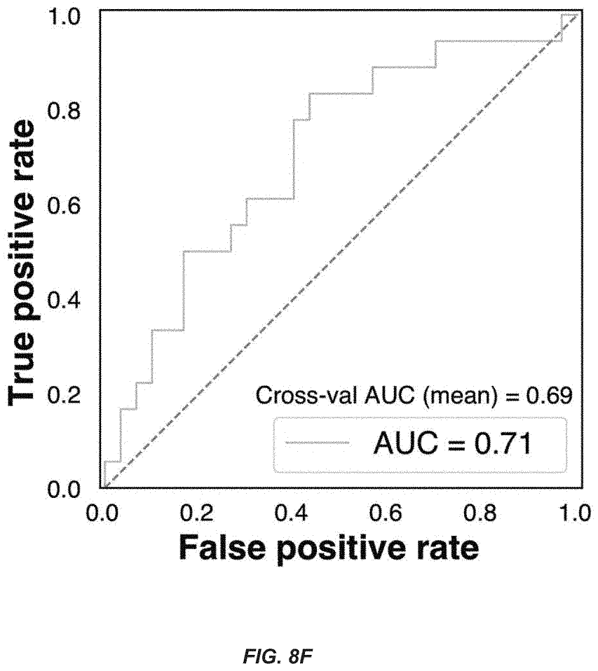

FIGS. 8A-F show statistical data identifying neoantigen burden scores across various subjects, in which the neoantigen burden score can be predictive of responsiveness of subjects treated with immunotherapies. FIG. 8A shows a set of box plots corresponding to a comparison of neoantigen burden scores between a first group of subjects that responded to immunotherapies and a second group of subject that did not respond to the immunotherapies. In FIG. 8A, each boxplot covers the interquartile range (interquartile range) from 25th percentile at its lower bound to the 75th percentile at its upper bound, with median indicated by a horizontal line. The upper whisker includes the largest value within 1.5× interquartile range above the 75th percentile. The lower whisker includes the smallest value within 1.5× interquartile range below 25th percentile. It was found that neoantigen burden is significantly higher in responding subjects compared to nonresponding subjects (n=51; MWW; P=0.016). FIG. 8B shows a set of box plots corresponding to a comparison of neoantigen burden scores of subject groups in the validation cohort (e.g., responsive subjects, non-responsive subjects). The data from the validation cohort in FIG. 8B confirms that subjects who responded to therapy presenting significantly higher neoantigen burden (MWW; P=0.021).

Other types of experimental data also indicate that higher neoantigen burden score is associated with responsiveness to immunotherapies. FIG. 8C shows a line plot that identifies a comparison of progression-free survival probability between a first group identified to have high neoantigen burden and a second group identified to have low neoantigen burden. As shown in FIG. 8C, significantly longer progression-free survival was observed in subjects with high neoantigen burden when compared to those with low neoantigen burden (two-sided KM log-rank test; P=0.002). FIG. 8D shows a line plot line plot that identifies a comparison of progression-free survival probability between subject groups in the validation cohort, and FIG. 8E shows a line plot that identifies a comparison of overall survival rate between subject groups in the validation cohort. Although FIG. 8D shows that progression-free survival of subject with high neoantigen burden was not significantly longer than those with low neoantigen burden in the validation cohort (two-sided KM log-rank test, P=0.085), FIG. 8E shows marked improvements to overall survival were observed in subjects with high neoantigen burden (two-sided KM log-rank test, P=0.005).

FIG. 8F shows a receiver operating characteristic curve that identifies performance levels of the neoantigen burden score model. As shown in FIG. 8F, the area under curve value for the neoantigen burden score model was 0.71 and the cross-validation area under curve value (mean) was 0.69 (log-likelihood ratio P=0.0329).

D. Genomic Metrics

1. Mutation Characteristics

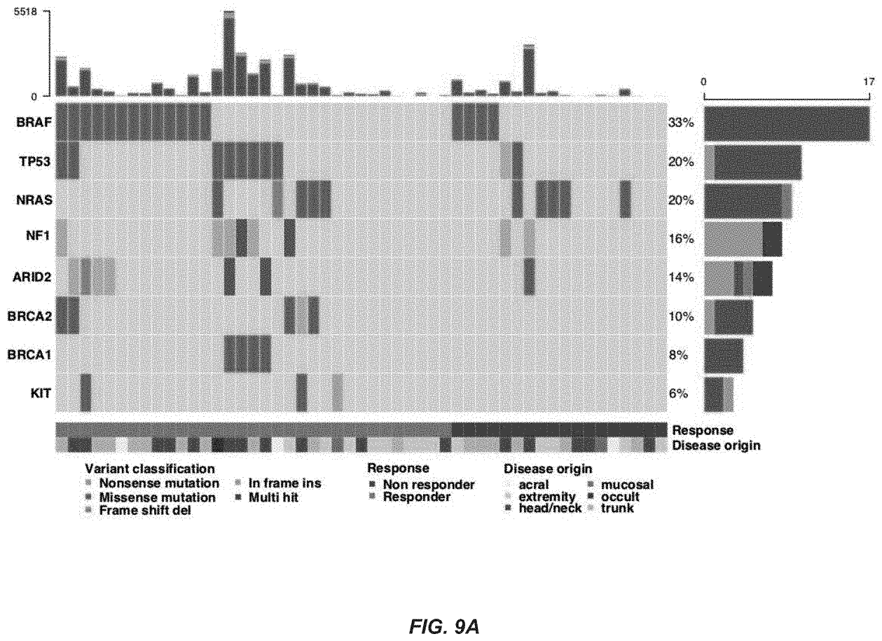

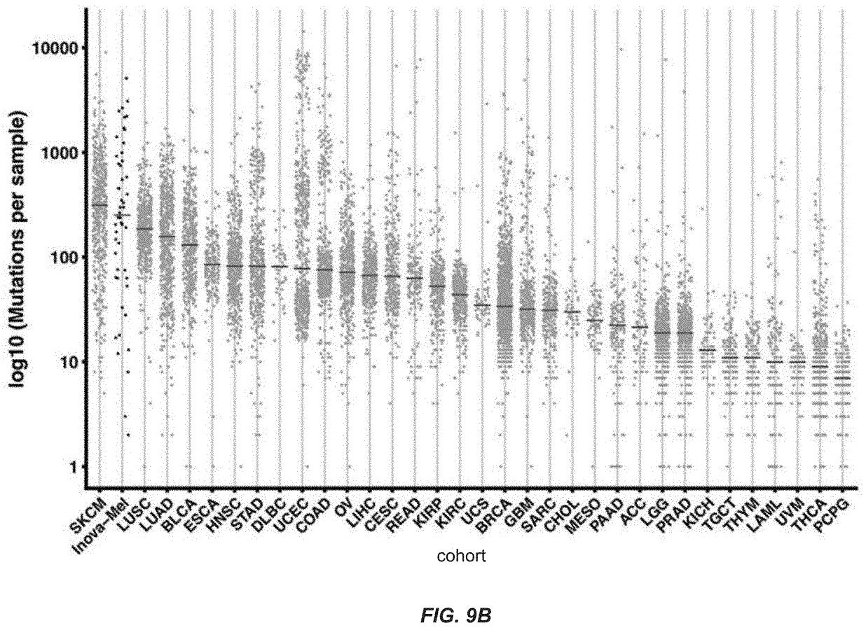

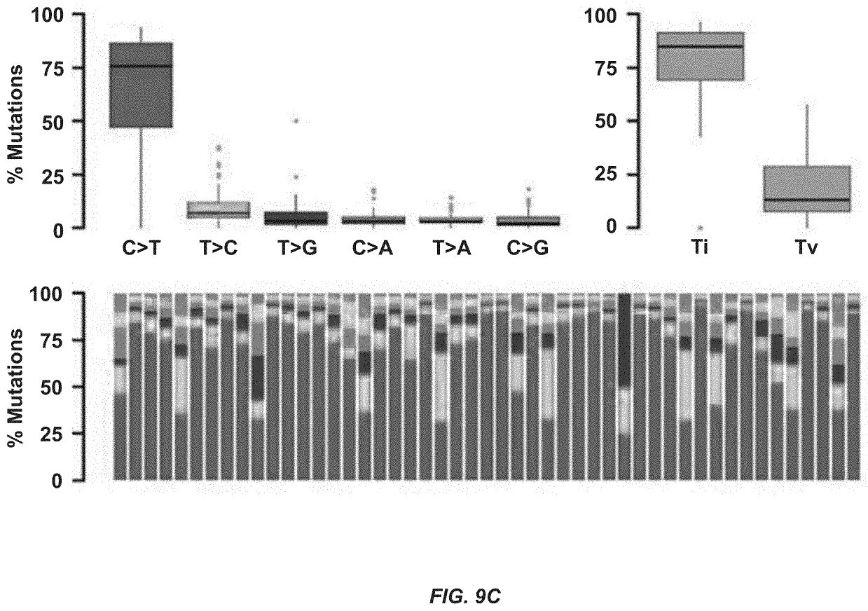

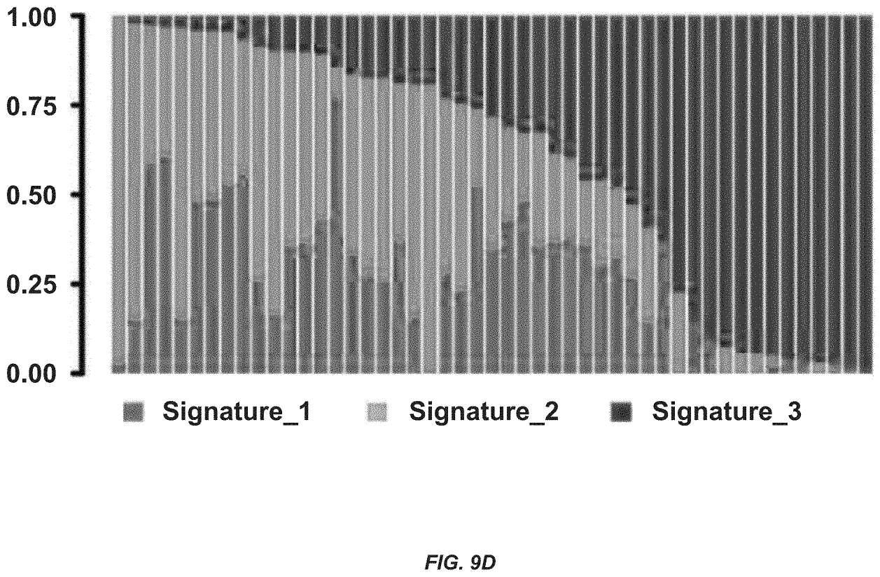

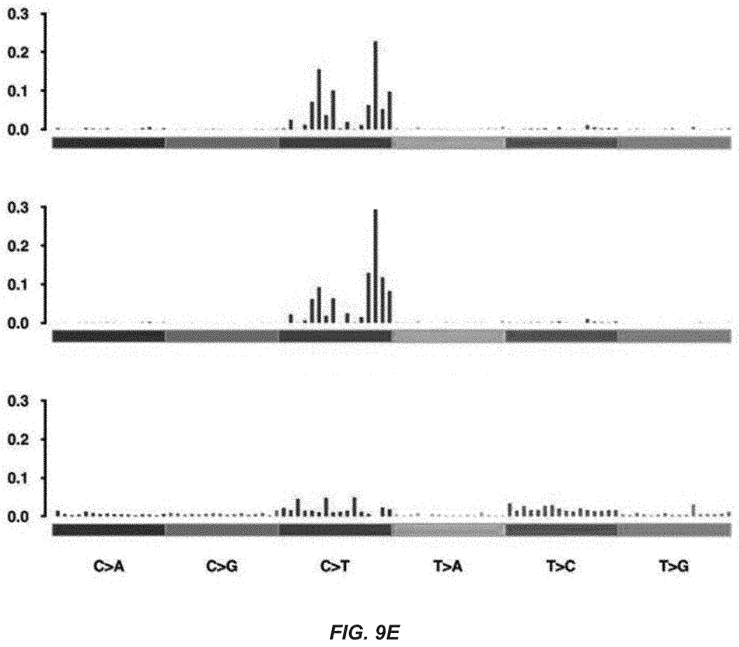

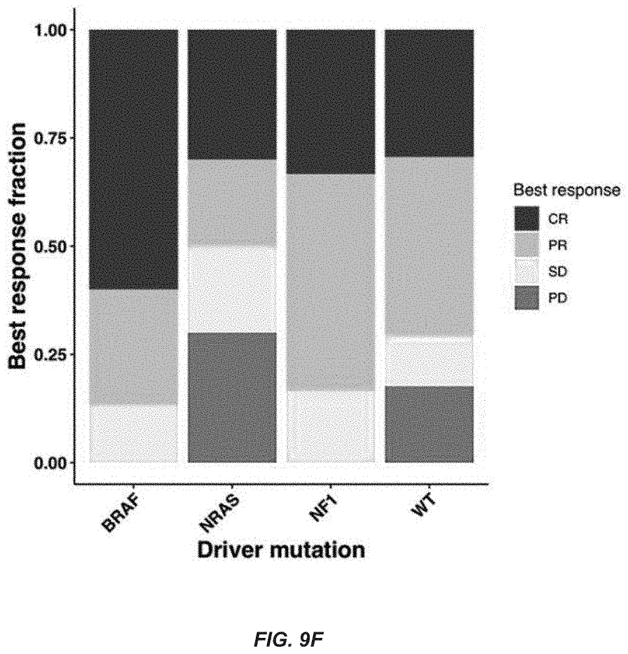

In addition to the transcriptomic data, genomic data was generated for each subject in the discovery cohort. From the genomic data, various genomic metrics were generated. FIG. 9A-F show statistical data that identify one or more characteristics relating to mutations present in each subject sample of the discovery cohort. FIG. 9A shows identifies mutations in various genes of subjects receiving anti-PD-1 therapy. In FIG. 9A, top box plot represents mutational load. Tiled plot shows mutated genes (rows) by sample (columns), with tile color indicating mutation type. The box plot to the right represents the number of subjects with mutations in the specified gene, colored to indicate mutation type. Under the tiled plot, the first line represents therapeutic response, as either response (partial or complete response; dark green; n=33), or non-response (black; n=18).

In FIG. 9A, median nonsynonymous tumor mutational burden was 4.07 mutations/MB (interquartile range, 0.95-12.455). This genomic metric appears to be consistent with values observed in known datasets. For example, FIG. 9B shows an amount of mutations identified in each sample across various datasets. Levels of mutational burden in the discovery cohort are comparable to those in TCGA-SKCM dataset (melanoma). In FIG. 9B, each dot represents a sample, with red horizontal lines at the median numbers of mutations in each cancer type. The (log scaled) vertical axis shows the number of mutations per sample.

FIG. 9C shows a set of box plots that identify an amount of mutations for each type of single-nucleotide variants and a bar graph showing a distribution of types of single-nucleotide variants for each subject in the discovery cohort. In FIG. 9C, single-nucleotide variants were classified as either transitions or transversions (n=49). Left boxplot shows overall distribution of six different substitution types, while right boxplot shows distribution of transitions (Ti) and transversions (Tv). As shown in FIG. 9C, C>T transitions appear to form the bulk of identified single-nucleotide variants (76%).

FIG. 9D shows a bar graph identifying a distribution of three mutational signatures for each subject in the discovery cohort. Signatures were extracted by decomposing a matrix of nucleotide substitutions, classified into 96 substitution classes based on bases immediately surrounding the mutated base, resulting in three primary signatures within the cohort. According to FIG. 9D, the most commonly identified driver mutation occurred in BRAF, in 33% of subjects, followed by 20% NRAS and 16% NF1 in the population. FIG. 9E shows a distribution of mutations of the three primary signatures. Extracted signatures were compared to previously validated signatures. Signature 1 and 2 in the discovery cohort are most similar to a UV signature, while the third signature most closely associated with a signature of unknown etiology. As shown in FIG. 9E, mutational signatures found in the discovery cohort most strongly associate with UV-induced DNA damage.

FIG. 9F shows a bar graph that identifies, for each driver mutation associated with a particular tumor (e.g., BRAF, NRAS), a distribution of subjects corresponding to various levels of responsiveness to immunotherapies. For example, responders were defined as complete response (CR) or partial response (PR). Non-responders were defined as stable disease (SD) or progressive disease (PD). Driver mutation can refer to a gene alteration that gives cancer cells a fundamental growth advantage for its neoplastic transformation. In FIG. 9F, subjects harboring BRAF mutated tumors were more likely to positively respond to therapy (n=47; Exact binomial test; P=0.0258). Response rate for the different genomic subtypes did not significantly vary from the expected response rate. The elevated number of progressive disease for WT gene in subjects likely arises from the reduced frequency of BRAF, which are typically observed at higher rates.

2. Tumor Mutational Burden

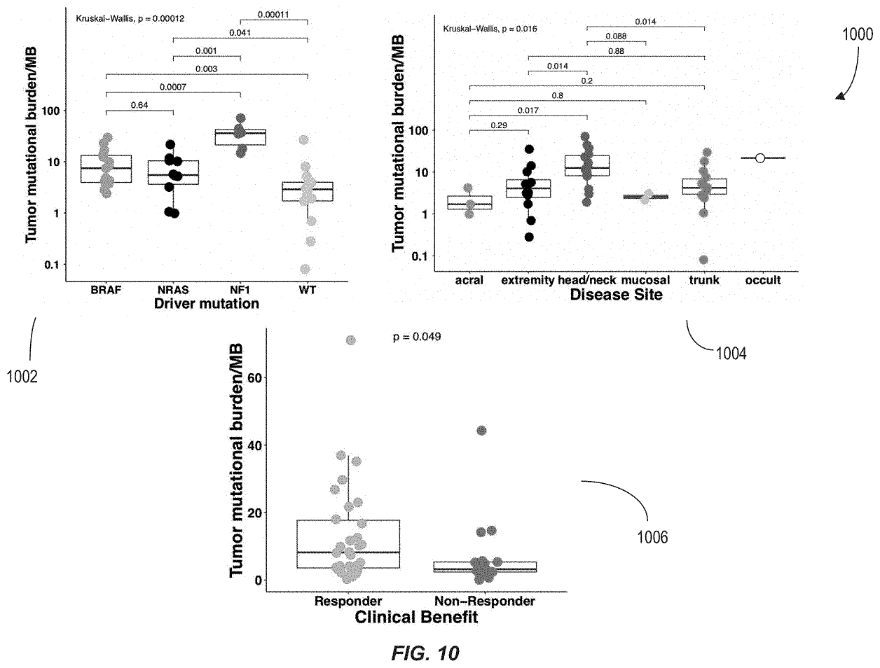

FIG. 10 shows sets of box plots 1000 that identify tumor mutational burden across various driver mutations, disease sites, and subject groups. The box plots 1000 includes boxplots 1002, 1004, and 1006. The box plots cover the interquartile range (interquartile range) from 25th percentile at their lower bound to the 75th percentile at their upper bound, with median indicated by a horizontal line. The upper whisker includes the largest value within 1.5× interquartile range above the 75th percentile. The lower whisker includes the smallest value within 1.5× interquartile range below 25th percentile. The values corresponding to the tumor mutational burden were plotted on log 10 scale.

The box plots 1002 identify tumor mutational burden for each driver mutation. Tumor mutational burden varied significantly between tumors harboring different driver mutations (Kruskal—Wallis, P=0.00012). The box plots 1004 identify tumor mutational burden for each of the identified sites of disease origin for melanoma. The box plots 1004 show significant global variation of tumor mutational burden across different sites of disease origin, with significant variation found in comparison with melanomas originating in the head and neck (Kruskal—Wallis, P=0.016).

The box plots 1006 identify tumor mutational burden for a first group of subjects that responded to immunotherapy and a second group of subjects that did not respond to the immunotherapy. The comparison of tumor mutational burden in responding vs non-responding subjects revealed significant associations (MMW; P=0.049). However, the relatively small variance between tumor mutational burden in responding and non-responding subjects in this cohort could be due to the confounding effects of melanoma subtype, and varying tumor purity, as these measures have recently been shown to limit tumor mutational burden's effectiveness as a predictive biomarker. Thus, tumor mutational burden alone may not be able to accurately predict responsiveness to immunotherapies.

E. Composite Biomarker Score

As described herein, embodiments of the present disclosure recognize that alterations in the antigen presenting machinery that could interfere with neoantigen presentation. Taking into such data could improve the performance of predicting responsiveness to immunotherapies, as these alterations have been noted individually to impact subject response to immune checkpoint blockade. Accordingly, the composite biomarker score adjusts the neoantigen burden score to account for subject specific tumor alterations that could interfere with neoantigen presentation, including HLA mutations, HLA loss of heterozygosity, and B2M mutations.

1. Discovery Cohort

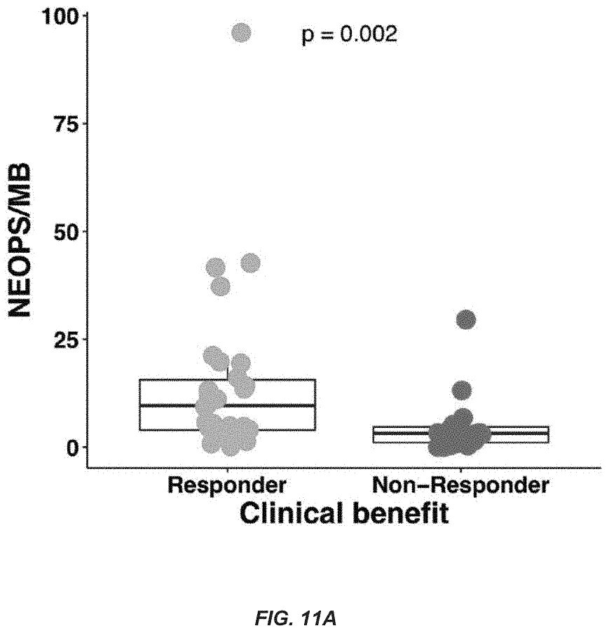

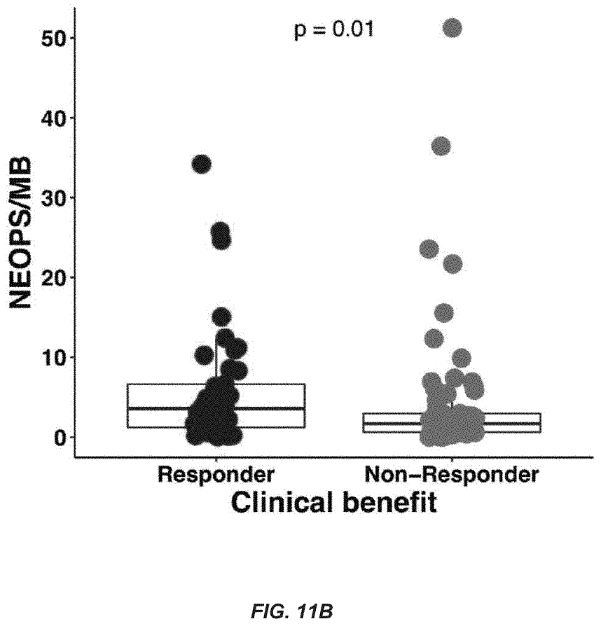

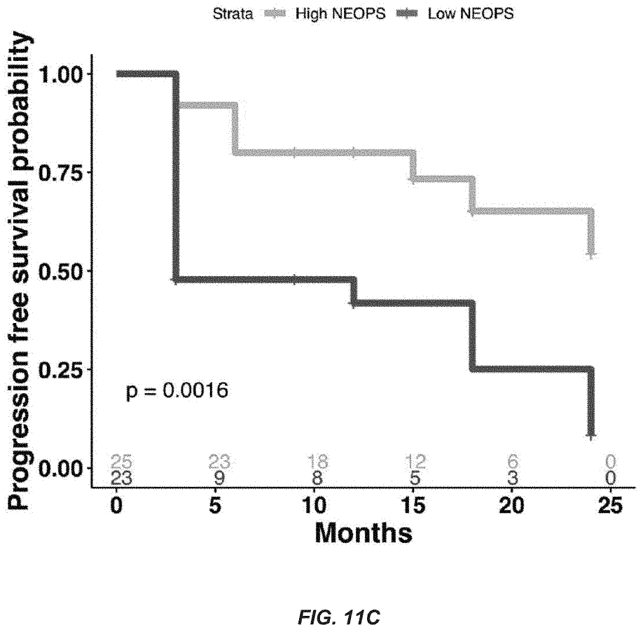

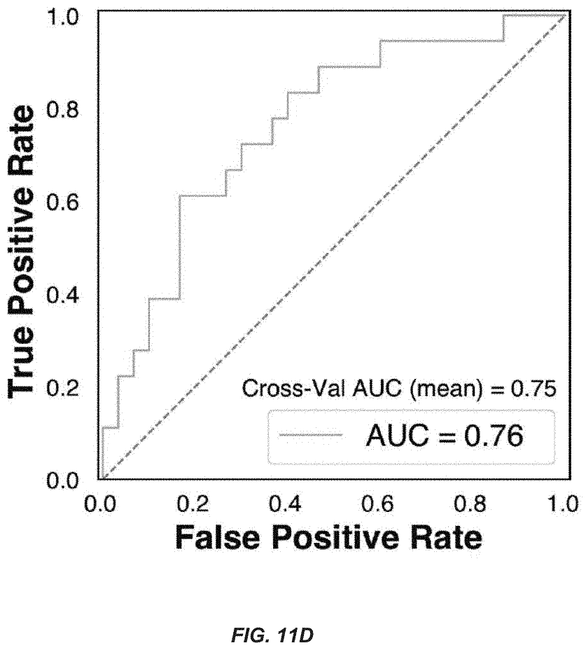

FIGS. 11A-D show statistical data identifying composite biomarker scores across various subjects, in which the composite biomarker scores indicate improved performance in predicting responsiveness of subjects treated with immunotherapies. In particular, FIGS. 11A-D show that composite biomarker score is more strongly associated with response to immunotherapies than neoantigen burden alone. For example, FIG. 11A shows a set of box plots corresponding to a comparison of composite biomarker scores between a first group of subjects that responded to immunotherapies and a second group of subject that did not respond to the immunotherapies. As shown in FIG. 11A, the composite biomarker score is significantly higher in responding subjects compared to non-responding subjects (n=51; MWW; P=0.002). Thus, the composite biomarker score resulted in improved prediction of therapy outcome, when compared to neoantigen burden. FIG. 11B shows a set of box plots corresponding to a comparison of composite biomarker scores of subject groups in the validation cohort (e.g., responsive subjects, non-responsive subjects). The data from the validation cohort in FIG. 11B confirms a similar result, in which subjects in the responsive group presenting significantly higher composite biomarker score than the non-responding subjects (n=110; MWW; P=0.010). With reference to FIGS. 11A-B, the corresponding box plots cover the interquartile range (interquartile range) from 25th percentile at its lower bound to the 75th percentile at its upper bound, with median indicated by a horizontal line. The upper whisker includes the largest value within 1.5× interquartile range above the 75th percentile. The lower whisker includes the smallest value within 1.5× interquartile range below 25th percentile.

FIG. 11C shows a line plot that identifies a comparison of progression-free survival probability between a first group identified to have high composite biomarker scores and a second group identified to have low composite biomarker scores. As shown in FIG. 11C, significantly longer progress-free survival was observed in subjects with high composite biomarker score when compared to those with low composite biomarker score (two-sided KM log-rank test; P=0.0016).

FIG. 11D shows a receiver operating characteristic curve that identifies performance levels of the composite biomarker score model. As shown in FIG. 11D, the composite biomarker model performs better than the neoantigen burden model: area under curve for the composite biomarker score increased to 0.76 from 0.71 and the cross-validation area under curve (mean) increased to 0.75 from 0.69 (log-likelihood ratio P=0.0057).

2. Validation Cohort

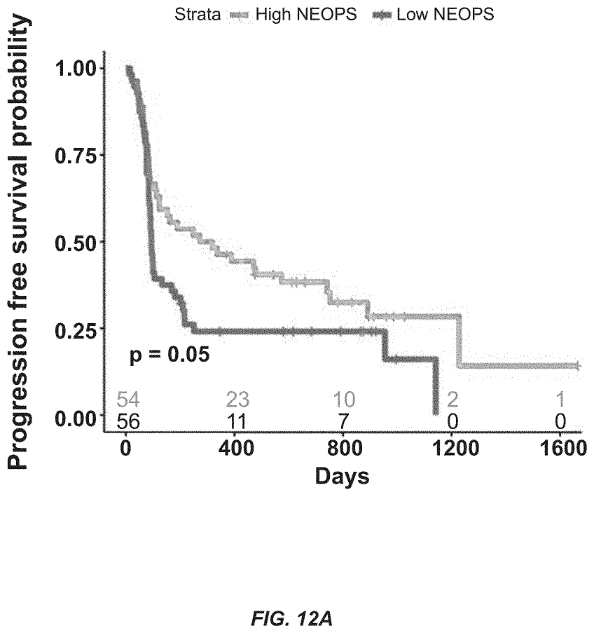

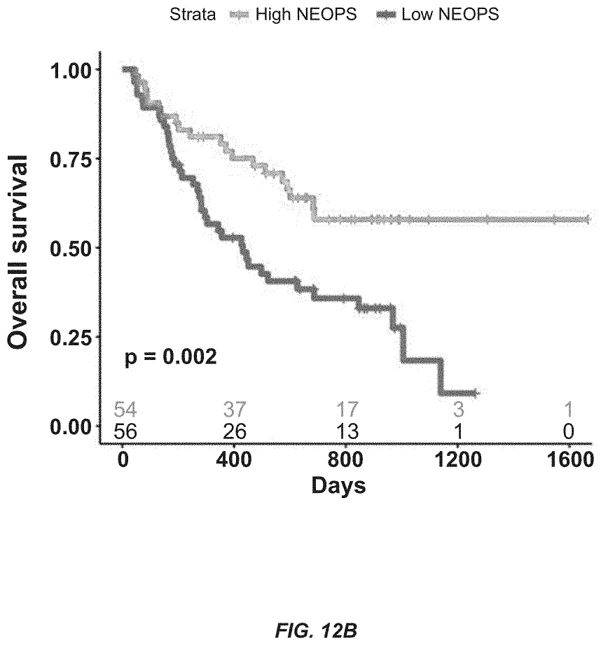

FIGS. 12A-B show statistical data identifying composite biomarker scores across various subjects, in which the composite biomarker scores indicate improved performance in predicting progression-free and overall survival rates of subjects in the cohort. In particular, the improvement of performance levels of the composite biomarker score was more noticeable in the validation cohort. FIG. 12A shows a line plot line plot that identifies a comparison of progression-free survival probability between subject groups in the validation cohort, and FIG. 12B shows a line plot that identifies a comparison of overall survival rate between subject groups in the validation cohort. In contrast to what was found for neoantigen burden score in the validation cohort, FIG. 12A shows that progression-free survival of subjects associated with high composite biomarker scores was significantly longer than subjects associated with low composite biomarker score (two-sided KM log-rank test, P=0.05). As also shown in FIG. 12B, greater significance was also achieved when analyzing overall survival, in which the overall survival rate was significantly longer in subjects associated with high composite biomarker score (two-sided KM log-rank test, P=0.002). The improvement with the composite biomarker score can be understood biologically with the finding that 23.5% of subjects in the discovery cohort, and 17.27% of subjects in the validation cohort had at least one mechanism potentially affecting antigen presentation, suggesting these features may frequently influence immune-system response to immunotherapies.

3. Mutations in HLA Genes Affecting the Composite Biomarker Score

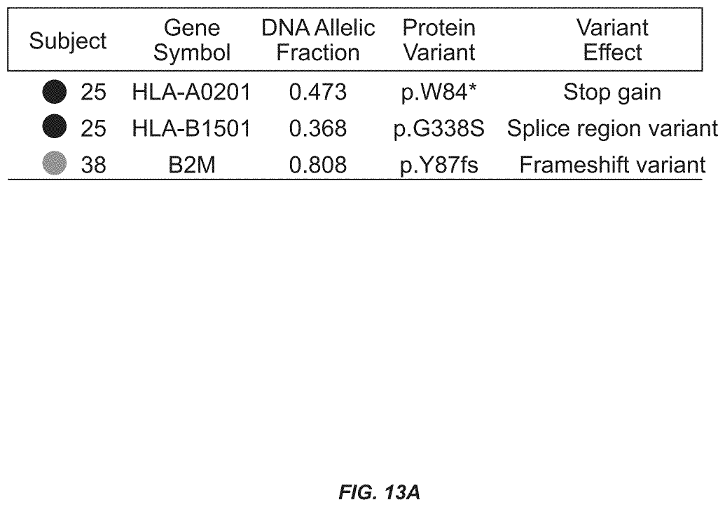

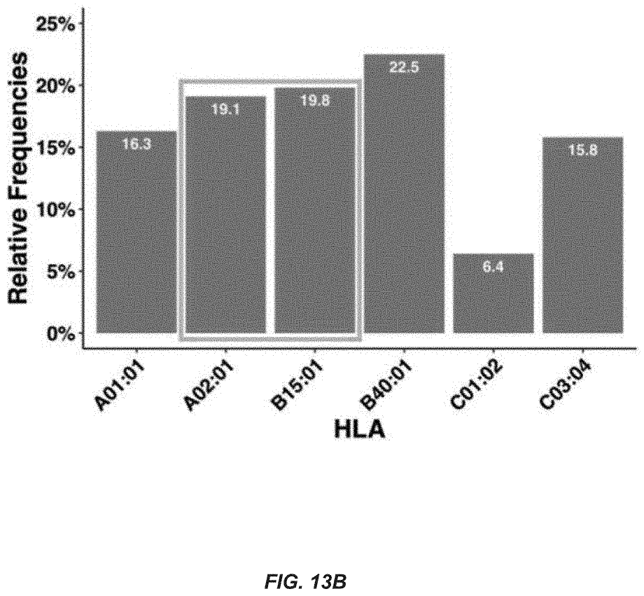

FIG. 13A-B show statistical data that identify somatic mutations to HLA genes that may contribute to a decreased probability of neoantigen presentation. In particular, a review of damaging HLA mutations across the discovery cohort revealed deleterious variants in many subjects. For example, FIG. 13A shows examples of somatic variants identified in samples of the discovery cohort. As shown in FIG. 13A, two distinct somatic HLA mutations were found in in subject 25, including a stop gain mutation in HLA-A02:01 and a splice region variant in HLA-B15:01 (allele fraction=0.473 and 0.368, respectively). These somatic mutations can lead to the loss of surface expression of HLA-A02:01 and possible misfolding of HLAB15:01. A damaging frameshift variant was detected in beta-2-microglobulin (B2M) in subject 38, possibly impairing all MHC class I presentation in that subject.

FIG. 13B shows a bar graph that identifies relative frequencies of neoantigens that are presented by respective HLA genes for subject 25 of the discovery cohort. In FIG. 13B, 38.9% of neoantigens (19.1% for A02:01; 19.8% for B15:01) in subject 25 were predicted to bind to the damaged HLA alleles, suggesting potentially severe impairment of neoantigen presentation. Of note, subject 25 was an outlier in the non-responding subjects, with much higher neoantigen burden, suggesting impaired neoantigen presentation beyond that which is captured in the composite biomarker score may be a contributing factor to immune checkpoint blockade resistance. In another outlier subject 38 (high neoantigen burden, non-responder), a damaging frameshift variant was detected in B2M at a high allelic fraction, also potentially impacting antigen presentation.

4. HLA Loss of Heterozygosity

HLA loss of heterozygosity was also examined in this cohort, as it can also impact neoantigen presentation. HLA loss of heterozygosity refers to an acquired resistance mechanism that facilitates immune escape by reducing capacity for presentation of tumor neoantigens to the immune system. As the process of HLA loss is governed by selective pressures within the tumor microenvironment, particularly at later stages of tumor evolution, it was hypothesized that within the cohort of late-stage melanoma subjects allele-specific HLA loss of heterozygosity could contribute to reduced therapeutic response despite apparent elevated neoantigen burden.

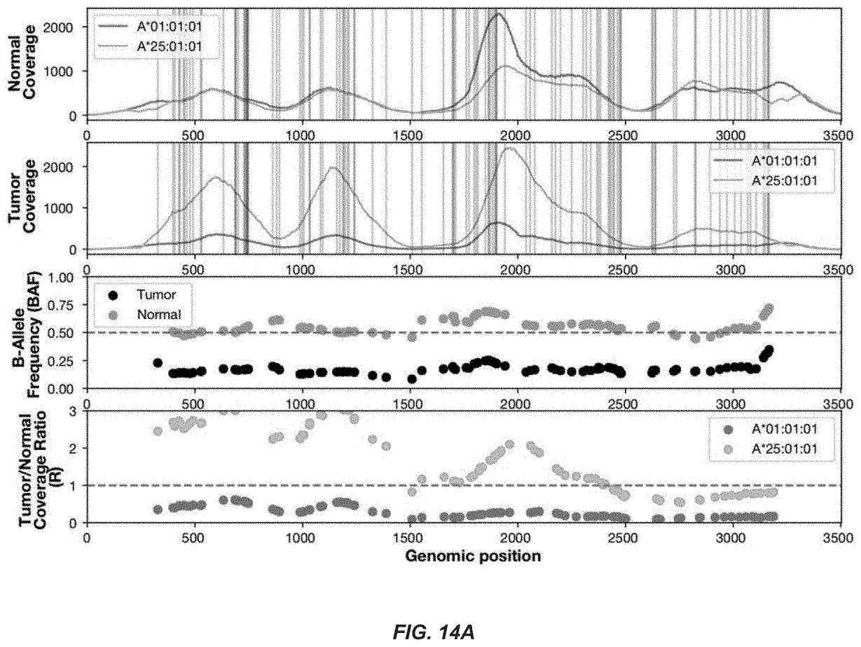

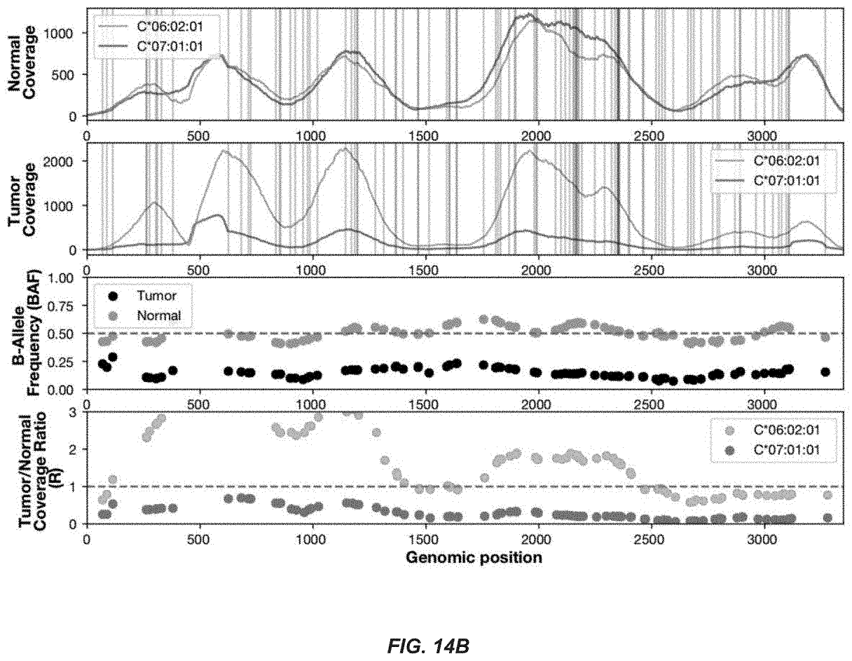

It was found that HLA loss of heterozygosity was the most prevalent form of HLA disruption, occurring in 19.6% of evaluable subjects (10/51), with three individuals presenting loss of heterozygosity across all non-homozygous HLAs. FIG. 14A-B shows examples of sets of panels that identify a comparison of HLA sequences between a normal sample and a corresponding tumor sample of a particular subject. For example, FIG. 14A shows a set of panels that identify a comparison of HLA-A sequences between the normal and tumor samples of the subject, and FIG. 14B shows a set of panels that identify a comparison of HLA-C sequences between the normal and tumor samples of the subject.

The panels of FIGS. 14A-B provide NGS sequence-based evidence for HLA loss of heterozygosity in HLA-A and HLA-C of subject 54 of the discovery cohort. HLA-B is not shown. The first row shows the raw read coverage of both homologous alleles in the normal sample. The second row shows the raw read coverage of both homologous alleles in the tumor sample. Both plots have vertical grey lines representing the positions of difference between the two alleles. Due to strict mapping parameters requiring all reads to map without mismatch, differences in coverage at the grey lines represent true differences in coverage between the alleles. The third panel shows the b-allele frequency from the normal sample (grey) and the tumor sample (black). The b-allele frequency in the tumor sample should be considered in light of the b-allele frequency in the normal sample because of primer hybridization differences between the alleles. The fourth panel shows the ratio in coverage between the tumor and normal samples for each allele. These values have been normalized by the tumor and normal read depth across the whole exome. The expected value with no copy number change is one, shown with a dashed grey line. Both the third and fourth panel only show data for the mismatch positions between the two alleles.

As shown in FIGS. 14A-B, matched normal tissue from the subject generally presents even allele specific coverage across HLA genes A and C. In contrast, tumor tissue from this subject exhibits broad imbalances in allele specific coverage spanning large portions of each HLA, with low levels of coverage in HLA-A01:01 and HLA-007:01. B-allele frequency (b-allele frequency) shows absolute difference from the normal. Consistently lower ratio of coverage is observed in the lost alleles (fourth rows in FIGS. 14A-B), which are predicted to present ˜54% of this subject's neoantigens, likely reducing capacity for presentation to the immune system.

### VII. Process for Generating a Composite Biomarker Score

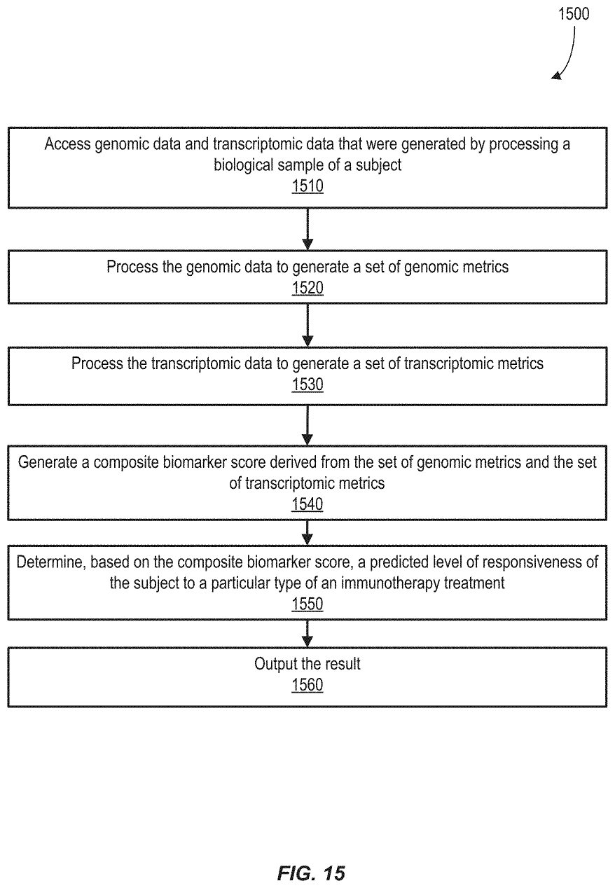

FIG. 15 includes a flowchart 1500 illustrating an example of a method of generating a composite biomarker score, according to some embodiments. Operations described in flowchart 1500 may be performed by, for example, a computer system implementing one or more operations for generating a composite biomarker score based on transcriptomic and genomic metrics. Although flowchart 1500 may describe the operations as a sequential process, in various embodiments, many of the operations may be performed in parallel or concurrently. In addition, the order of the operations may be rearranged. An operation may have additional steps not shown in the figure. Furthermore, embodiments of the method may be implemented by hardware, software, firmware, middleware, microcode, hardware description languages, or any combination thereof. When implemented in software, firmware, middleware, or microcode, the program code or code segments to perform the associated tasks may be stored in a computer-readable medium such as a storage medium.

At operation 1510, An immunogenomics-analysis system accesses genomic data and transcriptomic data that were generated by processing a biological sample of a subject. In some instances, the biological sample includes one or more cancer cells. The genomic data can identify one or more DNA sequences in the biological sample, in which whole-exome sequencing can be performed to identify the one or more DNA sequences. The transcriptomic data can identify one or more RNA sequences in the biological sample, in which transcriptome sequencing can be used to identify the one or more RNA sequences. Additionally or alternatively, the genomic and the transcriptomic data can be generated from a sample pair that includes the biological sample and a reference biological sample of the subject, in which the reference biological sample does not include the one or more cancer cells.

At operation 1520, the immunogenomics-analysis system processes the genomic data to generate a set of genomic metrics. Each of the set of genomic metrics can represent one or more characteristics corresponding to a corresponding DNA sequence the one or more DNA sequences. In some instances, the set of genomic metrics include: (i) a quantitative or categorical metric that represents one or more characteristics for each of one or more somatic mutations in the one or more DNA sequences; (ii) a categorical metric that indicates whether a loss of heterozygosity has occurred in at least one human leukocyte antigen (HLA) gene of the biological sample; and (iii) a quantitative or categorical metric that represents a predicted tumor mutational burden. With respect to the HLA loss of heterozygosity, the corresponding categorical metric can be generated by applying the genomic data to an HLA-deletion-identification machine-learning model.

At operation 1530, the immunogenomics-analysis system processes the transcriptomic data to generate a set of transcriptomic metrics. Each of the set of transcriptomic metrics can represent one or more characteristics corresponding to a set of peptides that are translated from a corresponding RNA sequence of the one or more RNA sequences. In some instances, the set of transcriptomic metrics include: (i) a quantitative or categorical metric that represents a predicted neoantigen burden of the biological sample; (ii) a quantitative or categorical metric that represents one or more characteristics of each of one or more candidate neoantigens detected from the biological sample; (iii) a quantitative or categorical metric that represents one or more characteristics of each of one or more HLA proteins for which a loss of cell-surface presentation is detected; (iv) a quantitative or categorical metric that represents one or more characteristics corresponding to an HLA gene that encodes the one or more HLA proteins for which the loss of cell-surface presentation was detected; (v) a quantitative or categorical metric that represents an expression level of a sequence corresponding to an immune cell; and (vi) a quantitative or categorical metric that represents an expression level of one or more T-cell receptors detected from the biological sample. With respect to the HLA proteins for which a loss of cell-surface presentation is detected, the corresponding metric can be generated by applying the genomic and transcriptomic data to a neoantigen-presentation-prediction machine-learning model.

At operation 1540, the immunogenomics-analysis system generates a composite biomarker score derived from the set of genomic metrics and the set of transcriptomic metrics. In some instances, the immunogenomics-analysis system generates the composite biomarker score by: (i) weighting each genomic metric of the set of genomic metrics with a weight value determined based on a corresponding transcriptomic metric of the set of transcriptomic metrics; and (ii) generating the composite biomarker score using the weighted genomic metrics.

At operation 1550, the immunogenomics-analysis system determines, based on the composite biomarker score, a predicted level of responsiveness of the subject to a particular type of an immunotherapy treatment.

At operation 1560, the immunogenomics-analysis system outputs a result that corresponds to the predicted level of responsiveness of the subject. The result can be report that identifies, based on the predicted level of responsiveness of the subject to the particular treatment: (i) a treatment recommendation of the particular treatment; (ii) a recommendation to administer the particular treatment to the human subject; and/or (iii) a recommendation to not administer the particular treatment to the human subject. In some embodiments, the recommended treatment is administered to the human subject. Process 1500 terminates thereafter.

### VIII. Additional Considerations

While the present subject matter has been described in detail with respect to specific embodiments thereof, it will be appreciated that those skilled in the art, upon attaining an understanding of the foregoing may readily produce alterations to, variations of, and equivalents to such embodiments. Accordingly, it should be understood that the present disclosure has been presented for purposes of example rather than limitation, and does not preclude inclusion of such modifications, variations, and/or additions to the present subject matter as would be readily apparent to one of ordinary skill in the art. Indeed, the methods and systems described herein may be embodied in a variety of other forms; furthermore, various omissions, substitutions and changes in the form of the methods and systems described herein may be made without departing from the spirit of the present disclosure. The accompanying claims and their equivalents are intended to cover such forms or modifications as would fall within the scope and spirit of the present disclosure.

Unless specifically stated otherwise, it is appreciated that throughout this specification discussions utilizing terms such as “processing,” “computing,” “calculating,” “determining,” and “identifying” or the like refer to actions or processes of a computing device, such as one or more computers or a similar electronic computing device or devices, that manipulate or transform data represented as physical electronic or magnetic quantities within memories, registers, or other information storage devices, transmission devices, or display devices of the computing platform.

The system or systems discussed herein are not limited to any particular hardware architecture or configuration. A computing device can include any suitable arrangement of components that provide a result conditioned on one or more inputs. Suitable computing devices include multipurpose microprocessor-based computing systems accessing stored software that programs or configures the computing system from a general purpose computing apparatus to a specialized computing apparatus implementing one or more embodiments of the present subject matter. Any suitable programming, scripting, or other type of language or combinations of languages may be used to implement the teachings contained herein in software to be used in programming or configuring a computing device.

Embodiments of Some embodiments may be performed in the operation of such computing devices. The order of the blocks presented in the examples above can be varied—for example, blocks can be re-ordered, combined, and/or broken into sub-blocks. Certain blocks or processes can be performed in parallel.

Conditional language used herein, such as, among others, “can,” “could,” “might,” “may,” “e.g.,” and the like, unless specifically stated otherwise, or otherwise understood within the context as used, is generally intended to convey that certain examples include, while other examples do not include, certain features, elements, and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example.

The terms “including,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. The use of “adapted to” or “configured to” herein is meant as open and inclusive language that does not foreclose devices adapted to or configured to perform additional tasks or steps. Additionally, the use of “based on” is meant to be open and inclusive, in that a process, step, calculation, or other action “based on” one or more recited conditions or values may, in practice, be based on additional conditions or values beyond those recited. Similarly, the use of “based at least in part on” is meant to be open and inclusive, in that a process, step, calculation, or other action “based at least in part on” one or more recited conditions or values may, in practice, be based on additional conditions or values beyond those recited. Headings, lists, and numbering included herein are for ease of explanation only and are not meant to be limiting.

The various features and processes described above may be used independently of one another, or may be combined in various ways. All possible combinations and sub-combinations are intended to fall within the scope of the present disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed examples. Similarly, the example systems and components described herein may be configured differently than described. For example, elements may be added to, removed from, or rearranged compared to the disclosed examples.

What is claimed is:

1. A method comprising:
accessing genomic data and transcriptomic data that were generated by processing a biological sample of a subject, wherein:
the biological sample includes one or more cancer cells;
the genomic data identifies one or more DNA sequences in the biological sample; and
the transcriptomic data identifies one or more RNA sequences in the biological sample;
generating, based on the genomic data, a set of genomic metrics, wherein each of the set of genomic metrics represents one or more characteristics corresponding to a corresponding DNA sequence the one or more DNA sequences;
generating, based on the transcriptomic data, a set of transcriptomic metrics, wherein each of the set of transcriptomic metrics represents one or more characteristics corresponding to a set of peptides that are translated from a corresponding RNA sequence of the one or more RNA sequences;
identifying a composite biomarker score derived from the set of genomic metrics and the set of transcriptomic metrics;
determining, based on the composite biomarker score, a predicted level of responsiveness of the subject to a particular type of an immunotherapy treatment; and
outputting a result that corresponds to the predicted level of responsiveness of the subject.

2. The method of claim 1, wherein generating the set of genomic metrics comprises determining a quantitative or categorical metric that represents one or more characteristics for each of one or more somatic mutations in the one or more DNA sequences.

3. The method of claim 1, wherein generating the set of genomic metrics comprises determining a categorical metric that indicates whether a loss of heterozygosity has occurred in at least one human leukocyte antigen (HLA) gene of the biological sample.

4. The method of claim 3, wherein determining the metric that indicates whether the loss of heterozygosity has occurred comprises applying the genomic data to an HLA-deletion-identification machine-learning model to generate an output that corresponds to the metric indicating whether loss of heterozygosity has occurred.

5. The method of claim 1, wherein generating the set of transcriptomic metrics comprises determining a quantitative or categorical metric that represents a predicted neoantigen burden of the biological sample.

6. The method of claim 1, wherein generating the set of transcriptomic metrics comprises determining, based on the genomic data and the transcriptomic data, a quantitative or categorical metric that represents one or more characteristics of each of one or more candidate neoantigens detected from the biological sample.

7. The method of claim 1, wherein generating the set of transcriptomic metrics comprises generating a quantitative or categorical metric that represents one or more characteristics of each of one or more HLA proteins for which a loss of cell-surface presentation is detected.

8. The method of claim 7, wherein generating the set of transcriptomic metrics comprises generating, based on the transcriptomic data, a quantitative or categorical metric that represents one or more characteristics corresponding to an HLA gene that encodes the one or more HLA proteins for which the loss of cell-surface presentation was detected.

9. The method of claim 7, wherein generating the set of transcriptomic metrics comprises applying the genomic data and the transcriptomic data to a neoantigen-presentation-prediction machine-learning model to generate the quantitative or categorical metric that represents the one or more characteristics of each of the one or more HLA proteins.

10. The method of claim 1, wherein generating the set of transcriptomic metrics includes determining a quantitative or categorical metric that represents an expression level of one or more T-cell receptors detected from the biological sample.

11. The method of claim 1, wherein the biological sample was collected from a tumor of the subject, and wherein generating the set of transcriptomic metrics includes determining a quantitative or categorical metric that represents an expression level of a sequence corresponding to an immune cell.

12. The method of claim 1, wherein accessing the genomic data and transcriptomic data comprises using whole-exome sequencing to identify the one or more DNA sequences.

13. The method of claim 1, wherein accessing the genomic data and transcriptomic data comprises using transcriptome sequencing to identify the one or more RNA sequences.

14. The method of claim 1, wherein accessing the genomic data and transcriptomic data comprises generating the genomic and the transcriptomic data from the biological sample and a reference biological sample of the subject, wherein the reference biological sample does not include the one or more cancer cells.

15. The method of claim 1, wherein generating the composite biomarker score includes:
weighting each genomic metric of the set of genomic metrics with a weight value determined based on a corresponding transcriptomic metric of the set of transcriptomic metrics; and
generating the composite biomarker score using the weighted genomic metrics.

16. A system comprising:
one or more data processors; and
a non-transitory computer readable storage medium containing instructions which, when executed on the one or more data processors, cause the one or more data processors to perform one or more operations comprising:
accessing genomic data and transcriptomic data that were generated by processing a biological sample of a subject, wherein:
the biological sample includes one or more cancer cells;
the genomic data identifies one or more DNA sequences in the biological sample; and
the transcriptomic data identifies one or more RNA sequences in the biological sample;
generating, based on the genomic data, a set of genomic metrics, wherein each of the set of genomic metrics represents one or more characteristics corresponding to a corresponding DNA sequence the one or more DNA sequences;
generating, based on the transcriptomic data, a set of transcriptomic metrics, wherein each of the set of transcriptomic metrics represents one or more characteristics corresponding to a set of peptides that are translated from a corresponding RNA sequence of the one or more RNA sequences;
identifying a composite biomarker score derived from the set of genomic metrics and the set of transcriptomic metrics;
determining, based on the composite biomarker score, a predicted level of responsiveness of the subject to a particular type of an immunotherapy treatment; and
outputting a result that corresponds to the predicted level of responsiveness of the subject.

17. A computer-program product tangibly embodied in a non-transitory machine-readable storage medium, including instructions configured to cause one or more data processors to perform one or more operations comprising:
accessing genomic data and transcriptomic data that were generated by processing a biological sample of a subject, wherein:
the biological sample includes one or more cancer cells;
the genomic data identifies one or more DNA sequences in the biological sample; and
the transcriptomic data identifies one or more RNA sequences in the biological sample;
generating, based on the genomic data, a set of genomic metrics, wherein each of the set of genomic metrics represents one or more characteristics corresponding to a corresponding DNA sequence the one or more DNA sequences;
generating, based on the transcriptomic data, a set of transcriptomic metrics, wherein each of the set of transcriptomic metrics represents one or more characteristics corresponding to a set of peptides that are translated from a corresponding RNA sequence of the one or more RNA sequences;
identifying a composite biomarker score derived from the set of genomic metrics and the set of transcriptomic metrics;
determining, based on the composite biomarker score, a predicted level of responsiveness of the subject to a particular type of an immunotherapy treatment; and
outputting a result that corresponds to the predicted level of responsiveness of the subject.

18. The computer-program product of claim 17, wherein generating the set of transcriptomic metrics comprises determining a quantitative or categorical metric that represents a predicted neoantigen burden of the biological sample.

19. The computer-program product of claim 17, wherein generating the set of transcriptomic metrics comprises determining, based on the genomic data and the transcriptomic data, a quantitative or categorical metric that represents one or more characteristics of each of one or more candidate neoantigens detected from the biological sample.

20. The computer-program product of claim 17, wherein generating the set of transcriptomic metrics comprises generating a quantitative or categorical metric that represents one or more characteristics of each of one or more HLA proteins for which a loss of cell-surface presentation is detected.
